(19) United States
(12) Patent Application Publication
(10) Pub. No.: US 2025/0199170 A1
(43) Pub. Date: Jun. 19, 2025

(54) QUADRATURE EXCITATION AND FRESNEL FOCUSING OF ROW-COLUMN TRANSDUCER ARRAYS

(71) Applicant: DAXSONICS ULTRASOUND INC., HALIFAX (CA)

(72) Inventors: JEREMY ALASTAIR BROWN, HALIFAX (CA); CHARLES D. EMERY, FREMONT, CA (US); KATHERINE GWEN LATHAM, DARTMOUTH (CA)

(21) Appl. No.: 18/844,241

(22) PCT Filed: Mar. 7, 2023
(86) PCT No.: PCT/CA2023/050294

§ 371 Date: Sep. 5, 2024

Related U.S. Application Data
(60) Provisional application No. 63/317,448, filed on Mar. 7, 2022.

(51) Int. Cl. G01S 15/89; B06B 1/02; B06B 1/06; G01S 7/52

(52) U.S. Cl. CPC G01S 15/8925; B06B 1/0223; B06B 1/0629; G01S 7/52046; G01S 15/8927; B06B 2201/76

(57) ABSTRACT

A row-column ultrasound transducer array is controlled to perform excitation in the azimuth dimension with two sets of excitation signals in quadrature. The two sets of excitation signals are delivered such that adjacent transducer elements are simultaneously provided excitation signals in quadrature, or such that transmit events occur temporally in series for synthetic imaging, with each transmit event being generated using a respective set of excitation signals. Separate bias apertures are applied in the bias (elevation) direction for each set of excitation signals, such that elements driven according to one set of excitation signals are biased, in the elevation direction, according to one bias aperture, and the elements driven with quadrature excitation signals are biased according to another bias aperture. The bias apertures are selected such that their combination results in the generation of a Fresnel aperture with fine and controllable phase resolution beyond that of a conventional row-column transducer.

### CROSS-REFERENCE TO RELATED APPLICATION

This application claims priority to U.S. Provisional Patent Application No. 63/317,448, titled “QUADRATURE EXCITATION AND FRENSEL FOCUSING OF ROW-COLUMN TRANSDUCER ARRAYS” and filed on Mar. 7, 2022, the entire contents of which is incorporated herein by reference.

### BACKGROUND

The present disclosure relates to ultrasound therapy and ultrasound imaging. More particularly, the present disclosure relates to row-column ultrasound transducer arrays and applications thereof.

Systems that can electronically steer and focus an ultrasound beam in three dimensions offer many advantages in both diagnostic imaging and therapy. Unfortunately, these systems also introduce significant challenges that originate at the transducer. This is because it is the design of the transducer that enables electronic steering and focusing of ultrasound in three dimensions. Ultrasound arrays with fine element sampling and independent electronic control in two dimensions, azimuth and elevation, are required. The element sampling in a second dimension when compared a standard linear or phased array increases the number from N (elements in a linear array) to N squared. For example, a 128 element linear array would require 16,384 individual elements in a 2D array. The dramatic increase in elements and the required electrical connections in a concentrated region creates a formidable interconnect issue. In addition to this challenge, the size of a 2D array element is significantly smaller than a conventional linear array element. The size reduction increases the element electrical impedance since it is inversely proportional to element area. The higher electrical impedance causes a reduction in transmit sensitivity and receive signal-to-noise ratio.

One way to circumvent the exorbitant number of electrical connections is through the application of crossed electrode arrays. Although these transducers enable volumetric imaging, the design offers less than adequate volume acquisition rates because both dimensions at the array cannot be beamformed simultaneously and synthetic apertures must be applied. Furthermore, the cross-electrode design is inherently problematic for therapeutic applications in which a sufficient aperture size is required in transmit to generate a high enough intensity at the focus.

For example, one application of a crossed electrode design uses one set of electrodes for transmit and the orthogonal set of electrodes for receive. The combined point-spread-functions (PSF's) of the top and bottom electrodes yield a two-way response that is similar in resolution and clutter to a 2D array if the aperture is twice the size of the fully sampled 2D array.

A method that circumvents these issues is the application of a row-column (crossed-electrode) array that uses electrostrictive material or CMUTs with a Fresnel focus. A Fresnel lens, when applied to optics, is a solution to generating a very tight focus when using a large aperture. In these situations, the larger aperture is problematic because of the required lens thickness which introduces loss and assembly challenges (i.e. device weight). A Fresnel lens introduces regions of varying thickness and curvature to still deliver a tight focus at the intended depth. These techniques are applicable in ultrasound where significant losses due to lens thickness are mitigated through the application of a Fresnel lens.

Although a Fresnel lens has been demonstrated in a passive lens material, it is also possible to discretely model a Fresnel lens using pulse polarity in the piezoelectric material. Transducer arrays fabricated with CMUTs or electrostrictive ceramics are ideal for this method since the pulse polarity may be controlled by a DC bias. The focus in azimuth and elevation is separated as in a conventional transducer. In the azimuth dimension, the focus is controlled with fine time delay control. In the elevation dimension, the focus is controlled by varying the polarity and is based on the ideal solution. If using electrostrictive material such as PMN-PT, the polarity is controlled through a bias on the material. The electrostrictive material only shows piezoelectric behavior while a DC voltage is applied. A positive DC bias results in a 0 degrees phase (positive pulse polarity) and a negative DC bias results in a 180 degrees phase (negative pulse polarity). Two phases are sufficient to discretely model a lens along the bias dimension. The discrete Fresnel pattern replaces the fixed mechanical lens with an electronic lens that offers two phases 0 degrees and 180 degrees.

The row-column transducer using a discrete Fresnel aperture significantly reduces the number of electrical connections. For example, a 64 by 64 conventional 2D array has 4,096 electrical connections whereas a row-column design has 128 electrical connections that consist of 64 beamforming channels and 64 bias lines. The row-column transducer reduction in beamforming complexity and density of electrical connections makes it an attractive alternative to volumetric imaging.

Unfortunately, the row-column transducer that uses a Fresnel pattern in the bias dimension also has some drawbacks. One major deficiency is the inability to effectively steer and focus the beam in three dimensions. Part of this deficiency can be attributed to the independent focusing in the azimuth and elevation dimensions. The other part of the deficiency may be attributed to the poor approximation of the Fresnel pattern to the required delays. Indeed, the utilization of only two phases, 0 degrees and 180 degrees, limits the ability to focus coherently due to delay errors in the bias dimension. This increases the off-axis energy which reduces the image contrast or ability to create sufficient focal gain for therapeutic purpose. Another issue with Fresnel-based implementations is that the phase is only an approximation. A conventional 2D array used time delays to precisely focus in three dimensions. If phases are the only means of controlling the focusing, then the discrete solution is only applicable for one frequency. Furthermore, if the required phase is beyond 360 degrees, then this creates blurring along the beam axis because pulses are inadvertently arriving more than one wavelength ahead or behind other waves from the aperture.

Recently, techniques to thwart these deficiencies have been developed. For example, Latham showed that using sub-apertures along the bias dimension can be used to avoid the long multi-cycle pulses that occur at the focal point when the required delay is more than one wavelength. This showed that the axial resolution improved by a factor of four for steered applications [K. Latham, et. al., “Design and Preliminary Experimental Results for a High Frequency Crossed Electrode Phased Array, Based on a Reconfigurable Fresnel Lens”, 2016 IEEE IUS]. Similarly, Latham used Simultaneous Azimuth and Fresnel Elevation (SAFE) compounding to suppress secondary lobe levels by compounding different Fresnel patterns without loss of frame rate. The secondary lobe levels were decreased by over −20 dB in the two-way beam pattern depending on the number of compounded patterns used [K. Latham, et. al., “Fabrication and Performance of a 128-element Crossed-Electrode Relaxor Array, for a novel 3D Imaging Approach”, 2017 IEEE IUS].

### SUMMARY

A row-column ultrasound transducer array is controlled to perform excitation in the azimuth dimension with two sets of excitation signals in quadrature. The two sets of excitation signals are delivered such that adjacent transducer elements are simultaneously provided excitation signals in quadrature, or such that transmit events occur temporally in series for synthetic imaging, with each transmit event being generated using a respective set of excitation signals. Separate bias apertures are applied in the bias (elevation) direction for each set of excitation signals, such that elements driven according to one set of excitation signals are biased, in the elevation direction, according to one bias aperture, and the elements driven with quadrature excitation signals are biased according to another bias aperture. The bias apertures are selected such that their combination results in the generation of a Fresnel aperture with fine and controllable phase resolution beyond that of a conventional row-column transducer.

Accordingly, in a first aspect, there is provided a system for performing ultrasound imaging, the system comprising:
a row-column ultrasound transducer comprising:
   a two-dimensional array of ultrasound elements arranged along a plurality of rows and columns, each ultrasound element being capable of acoustic transduction when a bias is applied thereto, such that a phase of ultrasound waves emitted therefrom is dependent on a polarity of the bias;
   a set of signal conductive paths, each signal conductive path being in electrical communication with signal electrodes of ultrasound elements residing along a respective column of the two-dimensional array; and
   a set of bias conductive paths, each bias conductive path being in electrical communication with bias electrodes of ultrasound elements residing along a respective row of the two-dimensional array; and
control and processing circuitry operatively coupled to the signal conductive paths and the bias conductive paths, the control and processing circuitry comprising at least one processor and associated memory, the memory comprising instructions executable by the processor to perform, in a selected temporal order, a set of synthetic transmit and receive operations comprising:
   a first transmit operation and first receive operation performed by:
      while applying a first transmit bias aperture to the bias conductive paths, delivering a first set of transmit signals to the signal conductive paths; and
      while applying a first receive bias aperture to the bias conductive paths, receiving a first set of receive signals from the signal conductive paths;
   a second transmit operation and second receive operation performed by:
      while applying the first transmit bias aperture to the bias conductive paths, delivering a second set of transmit signals to the signal conductive paths, the second set of transmit signals being generated according to a driving waveform that is in phase with a corresponding driving waveform employed to generate the first set of transmit signals;
      while applying a second receive bias aperture to the bias conductive paths, receiving a second set of receive signals from the signal conductive paths; and
      applying a time delay to the second set of received signals, the time delay corresponding to a phase delay of pi/2
   a third transmit operation and third receive operation performed by:
      while applying a second transmit bias aperture to the bias conductive paths, delivering a third set of transmit signals to the signal conductive paths, the third set of transmit signals being generated according to a driving waveform that is phase shifted, relative to a corresponding driving waveform employed to generate the first set of transmit signals, by pi/2; and
      while applying the first receive bias aperture to the bias conductive paths, receiving a third set of receive signals from the signal conductive paths; and
   a fourth transmit operation and fourth receive operation performed by:
      while applying the second transmit bias aperture to the bias conductive paths, delivering a fourth set of transmit signals to the signal conductive paths, the fourth set of transmit signals being generated according to a driving waveform that is phase shifted, relative to a corresponding driving waveform employed to generate the first set of transmit signals, by pi/2;
      while applying the second receive bias aperture to the bias conductive paths, receiving a fourth set of receive signals from the signal conductive paths; and
      applying a time delay to the fourth set of received signals, the time delay corresponding to a phase delay of pi/2;
   summing the first, second, third and fourth sets of receive signals to obtain a summed set of receive signals; and
   beamforming the summed set of receive signals in the azimuth dimension to generate an image;
   wherein the first set of transmit signals, the second set of transmit signals, the third set of transmit signals and the fourth set of transmit signals are selected such that the resulting plurality of ultrasound pulses generated therefrom spatially overlap with each other in a far field region;
   wherein the first transmit bias aperture and the second transmit bias aperture are configured to generate a synthetic transmit Fresnel aperture when the set of synthetic transmit and receive operations are compounded; and
   wherein the first receive bias aperture and the second receive bias aperture are configured to generate a synthetic receive Fresnel aperture when the set of synthetic transmit and receive operations are compounded.

In some example implementations, the control and processing circuitry is configured such that the synthetic transmit Fresnel aperture and the synthetic receive Fresnel aperture are configured to generate different elevation foci.

In some example implementations, The system according to claim 1 wherein the control and processing circuitry is configured such that the synthetic transmit Fresnel aperture and the synthetic receive Fresnel aperture are configured to generate common elevation foci.

In some example implementations, the control and processing circuitry is configured such an amplitude associated with the synthetic transmit Fresnel aperture is equal for at least two rows of the synthetic transmit Fresnel aperture.

In some example implementations, the control and processing circuitry is configured such an amplitude associated with the synthetic receive Fresnel aperture is equal for at least two rows of the synthetic receive Fresnel aperture.

In some example implementations, the control and processing circuitry is configured such that the bias levels of the transmit bias apertures and the receive bias apertures are generated according to a discrete set of bias levels, the discrete set of bias levels comprising at least three distinct bias levels.

The control and processing circuitry may be configured such that the bias levels of the first transmit bias aperture and the bias levels of the second transmit bias aperture are obtained from a lookup table, the lookup table associating, for each phase range of a plurality of phase ranges, a suitable first transmit aperture bias value selected from the discrete set of bias levels and a suitable second transmit aperture bias level selected from the discrete set of bias levels, such that the synthetic transmit Fresnel aperture generated by the compounding of the transmit and receive operations approximates a desired transmit Fresnel aperture.

The control and processing circuitry may be configured such that the bias levels of the first receive bias aperture and the bias levels of the second receive bias aperture are obtained from a lookup table, the lookup table associating, for each phase range of a plurality of phase ranges, a suitable first receive aperture bias value selected from the discrete set of bias levels and a suitable second receive aperture bias level selected from the discrete set of bias levels, such that the synthetic receive Fresnel aperture generated by the compounding of the transmit and receive operations approximates a desired ideal receive transmit Fresnel aperture.

In some example implementations, the control and processing circuitry is configured such that the set of synthetic transmit and receive operations are performed in a sequence that minimizes switching between the bias apertures.

In some example implementations, the control and processing circuitry is configured such that the first set of transmit signals, the second set of transmit signals, the third set of transmit signals and the fourth set of transmit signals are generated according to a time-delay aperture for focusing the resulting plurality of ultrasound pulses generated therefrom along a selected azimuth image line.

In some example implementations, the control and processing circuitry is configured such that the first set of transmit signals, the second set of transmit signals, the third set of transmit signals and the fourth set of transmit signals are selected such that the resulting plurality of ultrasound pulses generated therefrom have respective wavefronts suitable for performing coherent compound imaging in the azimuth direction.

The set of synthetic transmit and receive operations may be a first set of synthetic transmit and receive operations, and the control and processing circuitry may be configured such that:
the following additional steps are performed one or more times:
   performing an additional set of synthetic transmit and receive operations employing an additional first set of transmit signals, an additional second set of transmit signals, an additional third set of transmit signals and an additional fourth set of transmit signals selected such that the resulting plurality of additional ultrasound pulses generated therefrom have respective wavefronts suitable for performing coherent compound imaging in the azimuth direction;
   wherein the resulting additional first, additional second, additional third and additional fourth sets of receive signals are summed with the first, second, third and fourth sets of receive signals to obtain the summed set of receive signals.

A focal location corresponding to at least one of the synthetic transmit Fresnel aperture and the synthetic receive Fresnel aperture may be different for at least two sets of synthetic transmit and receive operations.

The synthetic transmit Fresnel aperture and the synthetic receive Fresnel aperture corresponding to each set of synthetic transmit and receive operations may be Fresnel sub-apertures.

In some example implementations, the row-column ultrasound transducer comprises an electrostrictive material.

In some example implementations, the row-column ultrasound transducer is formed from an array of capacitive micromachined ultrasound transducer elements.

In another aspect, there is provided a system for generating focused ultrasound, the system comprising:
a row-column ultrasound transducer comprising:
   a two-dimensional array of ultrasound elements arranged in a diamond pattern defining a set of rows and columns, such that for a given transducer element, a row associated with the given transducer element extends along a first axis connecting a first pair of opposing vertices of the given transducer element, and a column associated with the given transducer element extends along a second axis connecting a second pair of opposing vertices of the given transducer element, each ultrasound element being capable of acoustic transduction when a bias is applied thereto, such that a phase of ultrasound waves emitted therefrom is dependent on a polarity of the bias;
   a set of signal conductive paths, each signal conductive path extending along a respective column of the two-dimensional array and being in electrical communication with signal electrodes of ultrasound elements of the respective column, the set of signal conductive paths comprising a set of odd signal conductive paths and a set of even signal conductive paths, wherein each even signal conductive path extends along a respective even column of the diamond pattern and each odd signal conductive path extends along a respective odd column of the diamond pattern; and
   a set of bias conductive paths, each bias conductive path extending along a respective row of the two-dimensional array and being in electrical communication with bias electrodes of ultrasound elements of the respective row, the set of bias conductive paths comprising a set of odd bias conductive paths and a set of even bias conductive paths, wherein each even bias conductive path extends along a respective even row of the diamond pattern and each odd bias conductive path extends along a respective odd row of the diamond pattern; and
control and processing circuitry operatively coupled to the set of signal conductive paths and the set of bias conductive paths, the control and processing circuitry comprising at least one processor and associated memory, the memory comprising instructions executable by the processor to perform a transmit operation comprising:
   applying a first bias aperture to the set of odd bias conductive paths and a second bias aperture to the set of even bias conductive paths, the first bias aperture comprising a set of first bias values and the second bias aperture comprising a corresponding set of second bias values; and
   while applying the first bias aperture and the second bias aperture, delivering, to the set of odd signal conductive paths and the set of even signal conductive paths, a set of transmit signals defined according to a time-delay aperture, wherein set of transmit signals are provided to the set of even signal conductive paths in quadrature;
the set of first bias values and the corresponding set of second bias values being configured such that the transmit operation results in the generation of a synthetic Fresnel aperture.

In another aspect, there is provided a system for generating focused ultrasound, the system comprising:
a row-column ultrasound transducer comprising:
   a two-dimensional array of ultrasound elements arranged along a plurality of rows and columns;
   within each row, each ultrasound element comprising a first sub-element and a second sub-element residing laterally adjacent to one another, each sub-element being capable of acoustic transduction when a bias is applied thereto, such that a phase of ultrasound waves emitted therefrom is dependent on a polarity of the bias, such that each column of the two-dimensional array comprises a first sub-column of first sub-elements and a second sub-column column of second sub-elements;
   a set of signal conductive paths, each signal conductive path being in electrical communication with signal electrodes of ultrasound elements residing along a respective sub-column of the two-dimensional array, such that the first sub-column and second sub-column within a given column are separately addressable by respective first and second signal conductive paths; and
   a set of bias conductive path pairs, each bias conductive path pair comprising a first bias conductive path and a second bias conductive path that both extend along or adjacent to a given row of the two-dimensional array such that the first bias conductive path is configured to apply a respective bias to the first sub-elements with the given row and the second bias conductive path is configured to apply a respective bias to the second sub-elements within the given row; and
control and processing circuitry operatively coupled to the signal conductive paths and the set of bias conductive path pairs, the control and processing circuitry comprising at least one processor and associated memory, the memory comprising instructions executable by the processor to perform a transmit operation comprising:
   applying a first bias aperture to the first bias conductive paths and a second bias aperture to the second bias conductive paths, the first bias aperture comprising a set of first bias values and the second bias aperture comprising a corresponding set of second bias values; and
   while applying the first bias aperture and the second bias aperture, delivering a set of transmit signals to the signal conductive paths according to a time-delay aperture, such that the time-delay aperture defines transmit beamforming time delays associated with each column of the two-dimensional array, and such that for a given column having a respective beamforming time delay associated therewith, the transmit signal delivered to the first sub-column and the second sub-column have a common temporal envelope temporally delayed according to the beamforming delay, and wherein a waveform phase of the second sub-column is shifted, relative to that of the first sub-column, by pi/2, the first sub-column and the second sub-column of a given column thereby being delivered beamformed transmit signals in quadrature;
the set of first bias values and the corresponding set of second bias values being configured such that the transmit operation results in the generation of a Fresnel aperture.

In some example implementations, the control and processing circuitry is configured such an amplitude associated with the Fresnel aperture is equal for at least two rows of the Fresnel aperture.

The control and processing circuitry may be configured such that the set of first bias values and the set of second bias values are generated according to a discrete set of bias levels, the discrete set of at bias levels comprising at least three bias levels.

The control and processing circuitry may be configured such that each first bias value of the first bias aperture and each corresponding second bias value of the second bias aperture are obtained by: generating a lookup table associating, for each phase range of a plurality of phase ranges, a suitable first bias value selected from the discrete set of bias levels and a suitable second bias level selected from the discrete set of bias levels, such that the transmit operation applying the suitable first bias value and the suitable second bias level to ultrasound elements within a given row of the two-dimensional array results in a phase that lies within the phase range.

In some example implementations, the row-column ultrasound transducer comprises an electrostrictive material.

In some example implementations, the row-column ultrasound transducer is formed from an array of capacitive micromachined ultrasound transducer elements.

In another aspect, there is provided a method of performing ultrasound imaging, the method comprising:
providing a row-column ultrasound transducer comprising:
   a two-dimensional array of ultrasound elements arranged along a plurality of rows and columns, each ultrasound element being capable of acoustic transduction when a bias is applied thereto, such that a phase of ultrasound waves emitted therefrom is dependent on a polarity of the bias;
   a set of signal conductive paths, each signal conductive path being in electrical communication with signal electrodes of ultrasound elements residing along a respective column of the two-dimensional array; and
   a set of bias conductive paths, each bias conductive path being in electrical communication with bias electrodes of ultrasound elements residing along a respective row of the two-dimensional array; and
performing, in a selected temporal order, a set of synthetic transmit and receive operations comprising:
   a first transmit operation and first receive operation performed by:
      while applying a first transmit bias aperture to the bias conductive paths, delivering a first set of transmit signals to the signal conductive paths; and
      while applying a first receive bias aperture to the bias conductive paths, receiving a first set of receive signals from the signal conductive paths;
   a second transmit operation and second receive operation performed by:
      while applying the first transmit bias aperture to the bias conductive paths, delivering a second set of transmit signals to the signal conductive paths, the second set of transmit signals being generated according to a driving waveform that is in phase with a corresponding driving waveform employed to generate the first set of transmit signals;
      while applying a second receive bias aperture to the bias conductive paths, receiving a second set of receive signals from the signal conductive paths; and
      applying a time delay to the second set of received signals, the time delay corresponding to a phase delay of pi/2
   a third transmit operation and third receive operation performed by:
      while applying a second transmit bias aperture to the bias conductive paths, delivering a third set of transmit signals to the signal conductive paths, the third set of transmit signals being generated according to a driving waveform that is phase shifted, relative to a corresponding driving waveform employed to generate the first set of transmit signals, by pi/2; and
      while applying the first receive bias aperture to the bias conductive paths, receiving a third set of receive signals from the signal conductive paths; and
   a fourth transmit operation and fourth receive operation performed by:
      while applying the second transmit bias aperture to the bias conductive paths, delivering a fourth set of transmit signals to the signal conductive paths, the fourth set of transmit signals being generated according to a driving waveform that is phase shifted, relative to a corresponding driving waveform employed to generate the first set of transmit signals, by pi/2;
      while applying the second receive bias aperture to the bias conductive paths, receiving a fourth set of receive signals from the signal conductive paths; and
      applying a time delay to the fourth set of received signals, the time delay corresponding to a phase delay of pi/2;
   summing the first, second, third and fourth sets of receive signals to obtain a summed set of receive signals; and
   beamforming the summed set of receive signals in the azimuth dimension to generate an image;
   wherein the first set of transmit signals, the second set of transmit signals, the third set of transmit signals and the fourth set of transmit signals are selected such that the resulting plurality of ultrasound pulses generated therefrom spatially overlap with each other in a far field region;
   wherein the first transmit bias aperture and the second transmit bias aperture are configured to generate a synthetic transmit Fresnel aperture when the set of synthetic transmit and receive operations are compounded; and
   wherein the first receive bias aperture and the second receive bias aperture are configured to generate a synthetic receive Fresnel aperture when the set of synthetic transmit and receive operations are compounded.

In some example implementations, the synthetic transmit Fresnel aperture and the synthetic receive Fresnel aperture are configured to generate different elevation foci.

In some example implementations, the synthetic transmit Fresnel aperture and the synthetic receive Fresnel aperture are configured to generate common elevation foci.

The amplitude associated with the synthetic transmit Fresnel aperture may be equal for at least two rows of the synthetic transmit Fresnel aperture.

The amplitude associated with the synthetic receive Fresnel aperture may be equal for at least two rows of the synthetic receive Fresnel aperture.

In some example implementations, the bias levels of the transmit bias apertures and the receive bias apertures are generated according to a discrete set of bias levels, the discrete set of bias levels comprising at least three distinct bias levels.

The bias levels of the first transmit bias aperture and the bias levels of the second transmit bias aperture may be obtained from a lookup table, the lookup table associating, for each phase range of a plurality of phase ranges, a suitable first transmit aperture bias value selected from the discrete set of bias levels and a suitable second transmit aperture bias level selected from the discrete set of bias levels, such that the synthetic transmit Fresnel aperture generated by the compounding of the transmit and receive operations approximates a desired transmit Fresnel aperture.

The bias levels of the first receive bias aperture and the bias levels of the second receive bias aperture may be obtained from a lookup table, the lookup table associating, for each phase range of a plurality of phase ranges, a suitable first receive aperture bias value selected from the discrete set of bias levels and a suitable second receive aperture bias level selected from the discrete set of bias levels, such that the synthetic receive Fresnel aperture generated by the compounding of the transmit and receive operations approximates a desired ideal receive transmit Fresnel aperture.

In some example implementations, the set of synthetic transmit and receive operations are performed in a sequence that minimizes switching between the bias apertures.

In some example implementations, the first set of transmit signals, the second set of transmit signals, the third set of transmit signals and the fourth set of transmit signals are generated according to a time-delay aperture for focusing the resulting plurality of ultrasound pulses generated therefrom along a selected azimuth image line.

In some example implementations, the first set of transmit signals, the second set of transmit signals, the third set of transmit signals and the fourth set of transmit signals are selected such that the resulting plurality of ultrasound pulses generated therefrom have respective wavefronts suitable for performing coherent compound imaging in the azimuth direction.

The set of synthetic transmit and receive operations may be first set of synthetic transmit and receive operations, and the following additional steps may be performed one or more times:
performing an additional set of synthetic transmit and receive operations employing an additional first set of transmit signals, an additional second set of transmit signals, an additional third set of transmit signals and an additional fourth set of transmit signals selected such that the resulting plurality of additional ultrasound pulses generated therefrom have respective wavefronts suitable for performing coherent compound imaging in the azimuth direction;
wherein the resulting additional first, additional second, additional third and additional fourth sets of receive signals are summed with the first, second, third and fourth sets of receive signals to obtain the summed set of receive signals.

A focal location corresponding to at least one of the synthetic transmit Fresnel aperture and the synthetic receive Fresnel aperture may be different for at least two sets of synthetic transmit and receive operations.

The synthetic transmit Fresnel aperture and the synthetic receive Fresnel aperture corresponding to each set of synthetic transmit and receive operations may be Fresnel sub-apertures.

In some example implementations, the row-column ultrasound transducer comprises an electrostrictive material.

In some example implementations, the row-column ultrasound transducer is formed from an array of capacitive micromachined ultrasound transducer elements.

In another aspect, there is provided a method of generating focused ultrasound, the method comprising:
providing a row-column ultrasound transducer comprising:
   a two-dimensional array of ultrasound elements arranged in a diamond pattern defining a set of rows and columns, such that for a given transducer element, a row associated with the given transducer element extends along a first axis connecting a first pair of opposing vertices of the given transducer element, and a column associated with the given transducer element extends along a second axis connecting a second pair of opposing vertices of the given transducer element, each ultrasound element being capable of acoustic transduction when a bias is applied thereto, such that a phase of ultrasound waves emitted therefrom is dependent on a polarity of the bias;
   a set of signal conductive paths, each signal conductive path extending along a respective column of the two-dimensional array and being in electrical communication with signal electrodes of ultrasound elements of the respective column, the set of signal conductive paths comprising a set of odd signal conductive paths and a set of even signal conductive paths, wherein each even signal conductive path extends along a respective even column of the diamond pattern and each odd signal conductive path extends along a respective odd column of the diamond pattern; and
   a set of bias conductive paths, each bias conductive path extending along a respective row of the two-dimensional array and being in electrical communication with bias electrodes of ultrasound elements of the respective row, the set of bias conductive paths comprising a set of odd bias conductive paths and a set of even bias conductive paths, wherein each even bias conductive path extends along a respective even row of the diamond pattern and each odd bias conductive path extends along a respective odd row of the diamond pattern; and
applying a first bias aperture to the set of odd bias conductive paths and a second bias aperture to the set of even bias conductive paths, the first bias aperture comprising a set of first bias values and the second bias aperture comprising a corresponding set of second bias values; and
while applying the first bias aperture and the second bias aperture, delivering, to the set of odd signal conductive paths and the set of even signal conductive paths, a set of transmit signals defined according to a time-delay aperture, wherein set of transmit signals are provided to the set of even signal conductive paths in quadrature;
wherein the set of first bias values and the corresponding set of second bias values are configured such that the transmit operation results in the generation of a synthetic Fresnel aperture.

In another aspect, there is provided a method of generating focused ultrasound, the method comprising:
providing a row-column ultrasound transducer comprising:
   a two-dimensional array of ultrasound elements arranged along a plurality of rows and columns;
   within each row, each ultrasound element comprising a first sub-element and a second sub-element residing laterally adjacent to one another, each sub-element being capable of acoustic transduction when a bias is applied thereto, such that a phase of ultrasound waves emitted therefrom is dependent on a polarity of the bias, such that each column of the two-dimensional array comprises a first sub-column of first sub-elements and a second sub-column column of second sub-elements;
   a set of signal conductive paths, each signal conductive path being in electrical communication with signal electrodes of ultrasound elements residing along a respective sub-column of the two-dimensional array, such that the first sub-column and second sub-column within a given column are separately addressable by respective first and second signal conductive paths; and
   a set of bias conductive path pairs, each bias conductive path pair comprising a first bias conductive path and a second bias conductive path that both extend along or adjacent to a given row of the two-dimensional array such that the first bias conductive path is configured to apply a respective bias to the first sub-elements with the given row and the second bias conductive path is configured to apply a respective bias to the second sub-elements within the given row; and
applying a first bias aperture to the first bias conductive paths and a second bias aperture to the second bias conductive paths, the first bias aperture comprising a set of first bias values and the second bias aperture comprising a corresponding set of second bias values; and
while applying the first bias aperture and the second bias aperture, delivering a set of transmit signals to the signal conductive paths according to a time-delay aperture, such that the time-delay aperture defines transmit beamforming time delays associated with each column of the two-dimensional array, and such that for a given column having a respective beamforming time delay associated therewith, the transmit signal delivered to the first sub-column and the second sub-column have a common temporal envelope temporally delayed according to the beamforming delay, and wherein a waveform phase of the second sub-column is shifted, relative to that of the first sub-column, by pi/2, the first sub-column and the second sub-column of a given column thereby being delivered beamformed transmit signals in quadrature;
wherein the set of first bias values and the corresponding set of second bias values are configured such that the transmit operation results in the generation of a Fresnel aperture.

In some example implementations, an amplitude associated with the Fresnel aperture is equal for at least two rows of the Fresnel aperture.

In some example implementations, the set of first bias values and the set of second bias values are generated according to a discrete set of bias levels, the discrete set of at bias levels comprising at least three bias levels.

Each first bias value of the first bias aperture and each corresponding second bias value of the second bias aperture may be obtained by:
generating a lookup table associating, for each phase range of a plurality of phase ranges, a suitable first bias value selected from the discrete set of bias levels and a suitable second bias level selected from the discrete set of bias levels, such that the transmit operation applying the suitable first bias value and the suitable second bias level to ultrasound elements within a given row of the two-dimensional array results in a phase that lies within the phase range.

In some example implementations, the row-column ultrasound transducer comprises an electrostrictive material.

In some example implementations, the row-column ultrasound transducer is formed from an array of capacitive micromachined ultrasound transducer elements.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1A shows a conventional two-dimensional ultrasound array with N×M connections.

FIG. 1B shows a conventional row-column ultrasound array with 2N connections.

FIGS. 2A and 2B show a single element from the row-column array with a size of one wavelength in elevation and bias dimensions, respectively.

FIGS. 3A and 3B show an element configuration that is subdiced/subdivided in the bias dimension.

FIGS. 4A and 4B show an element configuration that is subdiced/subdivided in the signal dimension.

FIGS. 5A and 5B show an element that is subdiced/subdivided in both the signal and bias dimensions.

FIGS. 6A and 6B show an element that is subdiced/subdivided in the signal dimension with two separate voltage lines.

FIG. 6C is a table showing possible effective phases for the configuration shown in FIG. 6B.

FIG. 6D is a table showing possible effective phases when electrostrictive apodization in applied.

FIG. 6E is a table showing assigned bias amplitudes for five bias values based on the ideal phase.

FIG. 6F is a table showing assigned bias amplitudes for seven bias values based on the ideal phase.

FIGS. 6G, 6H, 6I and 6J show example row-column ultrasound transducer arrays configured for performing quadrature excitation and Fresnel focusing.

FIGS. 6K and 6L show example row-column ultrasound transducer arrays configured for performing quadrature excitation and Fresnel focusing, in which the transducer elements are arranged in a diamond pattern.

FIG. 7A shows an example of an ideal Fresnel lens and the approximation of this lens created by changing polarity across elements.

FIG. 7B shows the resulting relatively poor two-way radiation pattern from a single Fresnel approximation.

FIGS. 8A and 8B show the cosine and sine apertures employed during synthetic quadrature excitation and Fresnel focusing using a conventional row-column transducer.

FIG. 8C shows a set of transducer rows in the elevation direction.

FIGS. 8D and 8E show bias values (A_N and B_N, scaled to unity) of example cosine and sine bias apertures that when separately and sequentially applied to the elevation electrodes with the respective quadrature azimuth excitation signals.

FIG. 8F shows the synthetic compounded phase profile that approximates the Fresnel phase profile generated using the bias apertures shown in FIGS. 8D and 8E.

FIG. 8G shows simulated acoustic radiation patterns from compounding a Fresnel lens using sequential sine and cosine pulses to achieve fine delay resolution. Patterns from 2 and 4 Fresnel compounds are compared to an ideal linear array.

FIGS. 9A, 9B and 9C show the four transmit and receive events employed during synthetic quadrature excitation and Fresnel focusing using a conventional row-column transducer.

FIGS. 10A, 10B, 10C and 10D show the timing sequence of the four transmit and receive events employed during synthetic quadrature excitation and Fresnel focusing using a conventional row-column transducer.

FIG. 10E is a table showing an example sequence for performing four pulse-echo events.

FIG. 11A shows a fully sampled 2D array.

FIG. 11B shows a conventional row-column transducer.

FIG. 11C shows the cosine and sine apertures employed during synthetic quadrature excitation and Fresnel focusing using a conventional row-column transducer.

FIG. 11D is a table summarizing the channel count for each aperture type for two different examples.

FIG. 12 shows simulated two-way pulse echoes from Fresnel patterns focused to slightly different spatial locations.

FIG. 13 shows simulated two-way radiation pattern for a Fresnel lens with 32 total compounds (left) compared to a dynamically beamformed linear array (right).

FIG. 14 shows an example system for performing quadrature excitation and Fresnel focusing.

FIGS. 15A, 15B, 15C, 15D, 15E and 15F are tables showing results from simulations presenting the focal gain for various array configurations, including synthetic quadrature excitation and Fresnel focusing.

FIG. 16 plots results from simulations presenting the focal gain for various array configurations, including synthetic quadrature excitation and Fresnel focusing.

FIG. 17 is a table summarizing the differences between the conventional row-column transducer configurations and the quadrature row-column configurations.

FIGS. 18A, 18B, 18C and 18D show an example set of four bias amplitude (α_M and β_M) combinations for transmit and receive for an on-axis focus for the 64 element, wavelength pitch simulated aperture. In each pane the transmit aperture is in blue and the receive aperture is in red.

FIGS. 19A and 19B show example simulation results showing a) the exact phase delays required for an on-axis focus and b) the 2D two-way radiation pattern produced by the aperture.

FIGS. 20A, 20B, 20C and 20D show two-way radiation patterns with an on-axis focus showing, 2D radiation patterns from a) an example single aperture and c) the combined results of the quadrature apertures and the pressure profile at the focal depth b) for each aperture and d) for the combined apertures.

FIGS. 21A, 21B, 21C and 21D plot two-way radiation patterns with an off-axis focus (10-degree steering) showing, 2D radiation patterns from a) an example single aperture and c) the combined results of the quadrature apertures and the pressure profile at the focal depth b) for each aperture and d) for the combined apertures.

FIGS. 22A and 22B show two-way radiation pattern profiles showing the effect of increasing the number of bias levels (2, 3, 5, continuous) for a focus a) on-axis and b) steered 10 degrees.

FIG. 23 is an illustration of the sliding aperture where light coloured elements are active and dark coloured elements are inactive.

FIGS. 24A, 24B, 24C and 24D show simulated results using 8 focal locations to create the 8 Fresnel patterns, a) a two-way radiation pattern for a single compound, b) a comparison of the radiation pattern profile for a single compound, 8 compounds and for perfect delays, c) a two-way radiation pattern for 8 compounds and d) a two-way radiation pattern for a perfect delay profile.

FIG. 25A, 25B, 25C and 25D plot simulated results using 4 focal locations and 2 sub-apertures for a total of 8 compounds, a) a two-way radiation pattern for a single compound, b) a comparison of the radiation pattern profile for a single compound, 8 compounds and for perfect delays, c) a two-way radiation pattern for 8 compounds and d) a two-way radiation pattern for a perfect delay profile.

FIG. 26A, 26B, 26C and 26D plot simulated results using 2 focal locations and 4 sub-apertures for a total of 8 compounds, a) a two-way radiation pattern for a single compound, b) a comparison of the radiation pattern profile for a single compound, 8 compounds and for perfect delays, c) a two-way radiation pattern for 8 compounds and d) a two-way radiation pattern for a perfect delay profile.

FIG. 27A shows simulated beam profiles for the diverging wave dimension at steering angles of 0, 15, and 30 degrees for 32 diverging wave compounds (cross section of the radiation pattern at a depth of 11 mm).

FIG. 27B shows the radiation pattern simulated for the elevation dimension at a zero-degree steering angle only.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

Unless defined otherwise, all technical and scientific terms used herein are intended to have the same meaning as commonly understood to one of ordinary skill in the art. Unless otherwise indicated, such as through context, as used herein, the following terms are intended to have the following meanings:

As described above, row-column transducers mitigate the excessive channel count in fully sampled 2D arrays used for volume imaging by independently addressing the focusing in the azimuth and elevation dimensions. For example, a fully sampled 64 by 64 2D array requires 4,096 electrical connections whereas a row-column design only requires 128electrical connections. When electrostrictive material is applied, this is accomplished by using standard electronic delays along one dimension and voltage biasing in the other dimension. The voltage biasing along this dimension offers two distinct phases: 0 degrees and 180 degrees.

Unfortunately, discretely modeling the necessary phasing in the bias dimension with only two phases has significant challenges for both ultrasound imaging and therapy. In ultrasound imaging, the use of only two phases for use in Fresnel-based focusing leads to poor beam control such that there is significantly higher clutter around the intended focus due to secondary lobes. This shows up as a reduction in image contrast when compared to a fully sampled 2D array, as the amount of energy off-axis reduces contrast when compared to a 2D array degrading detection. Furthermore, there is an inability to steer the beam because of the lack of electronic beam control in elevation.

Similarly, in ultrasound therapy, the inability to effectively concentrate the ultrasound energy at the therapy focus when using a row-column transducer yields poor focal gains, causing slower heating rates and greater safety issues in tissues away from the focus. The focal gain is significantly lower than a full 2D array and this requires either an increase in the device area which is intractable in some applications or an increase in the surface intensity which may not be achievable because of material limitations.

The present inventors realized that the limitations of conventional row-column transducer methods using Fresnel elevation focusing could be circumvented by employing quadrature (orthogonal) excitation in the signal (azimuth) dimension, with separate bias apertures for each of the two sets of quadrature excitation signals, to synthetically generate a composite Fresnel aperture with finer and controllable phase resolution. The inventors hypothesized that quadrature excitation of elements (columns) in azimuth (alternating cosine and sine functions) would facilitate improved elevation beam control because multiple phases are available locally between two adjacent elevation rows. In addition, the inventors realized that apodization of the poling voltage in elevation can be employed to yield a multiplicity of different phase angles in elevation which enables performance approaching that of a 2D array.

As will be shown below, such an approach, henceforth referred to as the “quadrature excitation Fresnel focusing” method, enables the generation of complex phase profiles across the elevation aperture that extend beyond the mere two phase values that were previously obtainable using the conventional approach. The increased phase resolution is facilitated by the use of separate sets of quadrature excitation signals that are delivered to the azimuth electrodes, in combination with the use of quadrature-excitation-specific bias (elevation) apertures that, when combined, generate the resulting finer-resolution phase profile in the elevation direction. As will be described below, the phase control that can be achieved in the bias dimension can be adjusted by selecting a suitable number of levels of amplitude modulation on the bias lines. For example, simulations presented in the examples below will demonstrate that contrast can improve by over 20 dB one-way for imaging with even better steer capabilities along the bias dimensions. Furthermore, focal gains have shown improvement by over 50% approaching that of a 2D array by introducing eight new phases through the orthogonal excitation in azimuth and amplitude modulation along the bias lines.

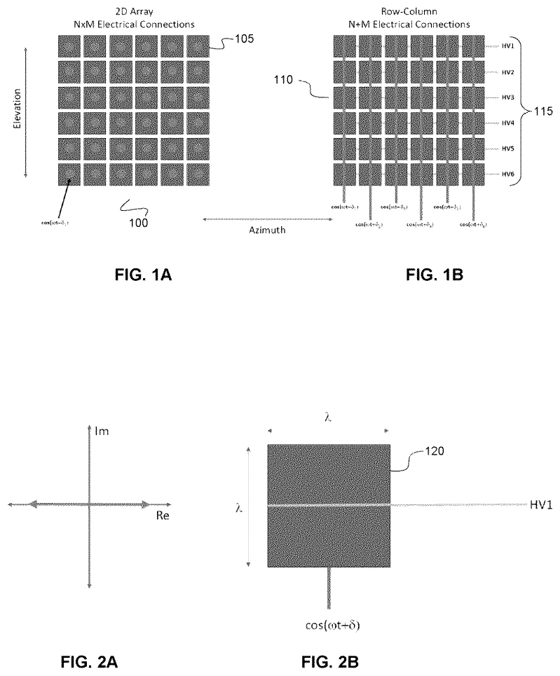

The improved quadrature excitation multiphasic methods of the present disclosure are described below after first considering the limitations of conventional 2D ultrasound arrays and conventional biphasic row-column arrays. Referring first to FIG. 1A, a top-level view of a conventional 2D array 100 is shown with N×M electrical connections. In this conventional design, each element 105 has separate electronic control which enables focusing and steering of the beam in three dimensions. The excitation on any given element in the 2D array is given by:

$$f ( t ) = {A}_{N , M} * cos ( 2 * π * {f}_{op} * t + {δ}_{N , M} ) \qquad ( 1 )$$

where A_{N,M} is the amplitude, f is the operation frequency in Hz, t is the time in seconds, and δ_{N,M} is the delay on the element in column N and row M for a focus in 3D space.

FIG. 1B shows the top view of a row-column transducer 110 using polarity in the elevation dimension to control the focus via the generation of two phase values across the elevation aperture 115. The excitation on any given element in the 2D array is given by:

$$f ( t ) = {B}_{M} * {A}_{N} * cos ( 2 * π * {f}_{op} * t + {δ}_{N} ) \qquad ( 2 )$$

$${B}_{M} = \{ + 1 , - 1 , 0 \} \qquad ( 3 )$$

where B_{M} is the polarity for the bias dimension and is based on the ideal phasing for the focus in depth and elevation at the operation frequency, A_{N} is the amplitude of the beamforming channel, and on is the delay for a focus in depth and azimuth. While the excitation voltage is normalized to unity in the present example, it will be understood that a suitable excitation voltage will depend on the specific application and transducer configuration. It is noted that even though there are three different bias values that are applied to the bias electrodes, they only result in the generation of two phases, since the zero bias value does not result in transducer actuation.

FIG. 2B shows a single element from the row-column array with a size of one wavelength in elevation and bias dimensions, respectively. This distance is determined by the wavelength of the operating frequency is water:

$${λ}_{element} = \frac{{c}_{H 2 O}}{{f}_{op}} \qquad ( 4 )$$

Unfortunately, one element in the row-column array only has two phase choices, as shown in FIG. 2A that illustrates the phase values residing on a real axis and imaginary axis. A vector that sits along the real axis is used to demonstrate that the phase of the element is either 0 degrees or 180 degrees. The present inventors, understanding the limitations of this approach, sought to develop an alternative excitation scheme that would result in the generation of a plurality of phase values.

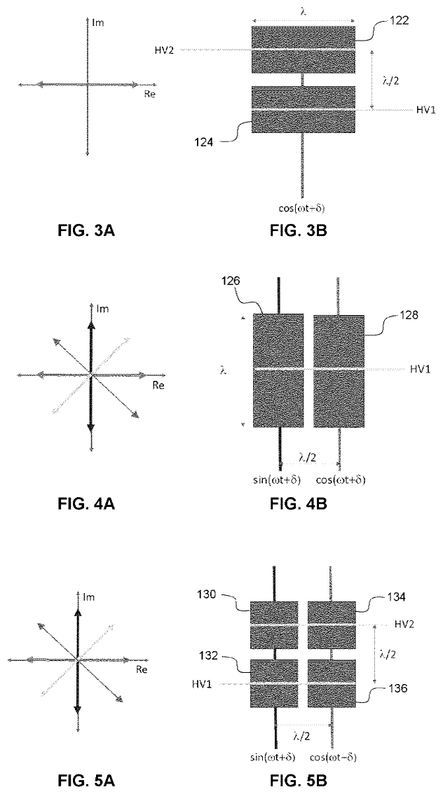

FIG. 3B shows an element configuration that is subdiced/subdivided in the bias (elevation) dimension into two sub-elements 122 and 124. This approach requires an additional high-voltage line, shown as HV2. Despite the addition of the second high-voltage line, this configuration still only offers two distinct phases, 0 degrees and 180 degrees, as shown in FIG. 3A. If the two sub-elements are simultaneously excited at 0 degrees and 180 degrees respectively, then the net result is a null vector since the two excitations cancel out each other.

FIG. 4B shows an element configuration that is subdiced/subdivided in the signal dimension into sub-elements 126 and 128. This requires an additional signal line such that two orthogonal excitations are possible within the same element. Only one bias line is connected in this configuration. The quadrature (orthogonal) excitations can be described as:

$$f ( t ) = {B}_{M} * {A}_{N} * sin ( 2 * π * {f}_{op} * t + {δ}_{N} ) \qquad ( 5 a )$$

$$f ( t ) = {B}_{M} * {A}_{N} * cos ( 2 * π * {f}_{op} * t + {δ}_{N} ) \qquad ( 5 b )$$

When considering the available phases within an element, since the sine and cosine elements in the bias dimension are not independent, there are still only two possibilities separated by 180 degrees since both the sine and cosine excitation are used simultaneously. If the high voltage line is positive (0 degrees), then the net phase is 45 degrees. If the high voltage line is negative (180 degrees), then the net phase is 225 degrees. This is diagramed in FIG. 4A with the additional vector shown on the real and imaginary axes. It is also possible to use a combination of a negative sine excitation and positive cosine excitation or a positive sine excitation and a negative cosine excitation. However, this still does not improve the available phase angles with one bias line. In this case, this case the available angles are still separated by 180 degrees (135 degrees and 315 degrees).

FIG. 5B shows an element that is subdiced/subdivided in both the signal and bias dimensions into sub-elements 130, 132, 134 and 136. Quadrature excitation is applied to the subdivided pair of signal sub-elements. In this example, the bias lines still cross both of the sine and cosine excitations applied to the signal sub-element elements. When comparing this scenario to that of the configuration shown in FIG. 4A, this approach still only yields two possible phases, namely 45 degrees and 225 degrees, as shown in FIG. 5A or 135 degrees and 315 degrees if a negative sine or negative cosine excitation is used. Furthermore, if the DC voltages of HV1 and HV2 are opposite, then the two excitations cancel each other out as in FIG. 3A.

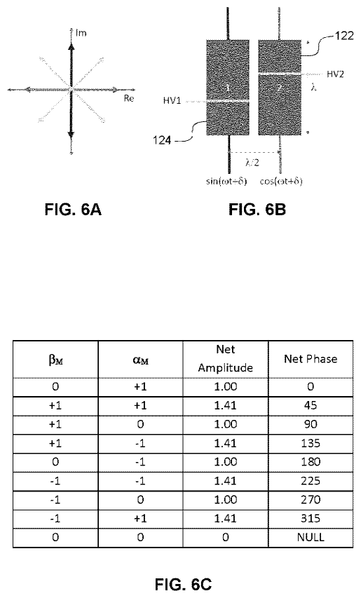

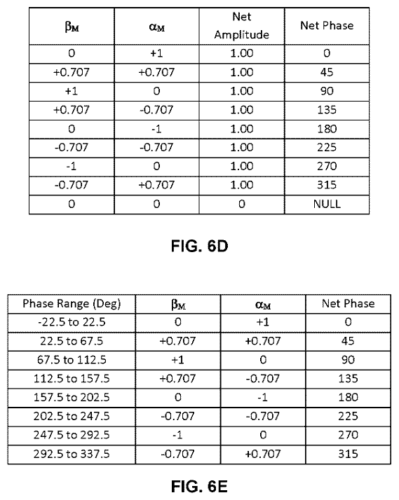

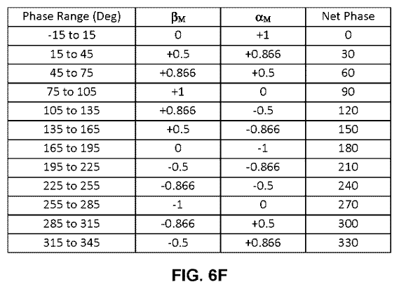

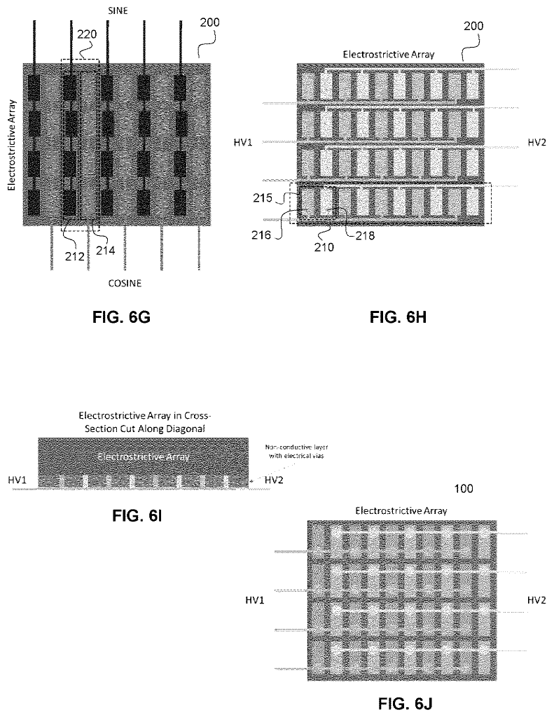

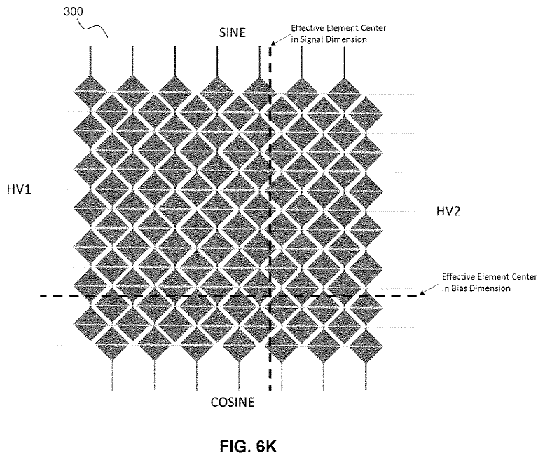

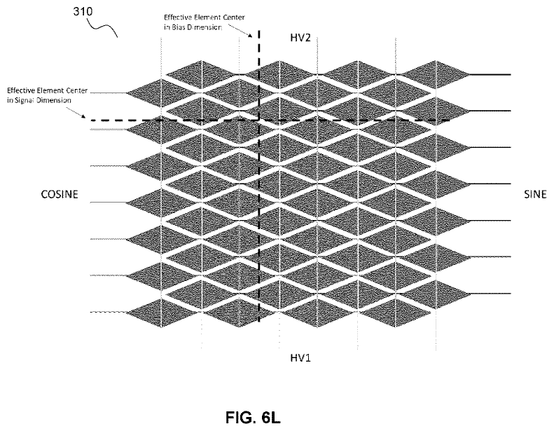

However, as illustrated in FIGS. 6A and 6B, if the element is subdiced/subdivided in the signal dimension into sub-elements 122 and 124 and two separate high voltage lines HV1 and HV2 are employed to set the polarity for the sub-elements, then instead of having just one high voltage line, finer phase control can be achieved under quadrature excitation of the sub-elements with different bias voltages applied to each sub-element. Accordingly, such an example embodiment can yield a significant advantage over the embodiments shown in the preceding figures.

This advantage can be appreciated by considering an example implementation involving the use of different combinations of three bias values applied to the high voltage lines (elevation electrodes). As will be shown below, in the case of the present example implementation, when only using three distinct bias values of 0, +V and −V, a total of eight different effective phases (resulting from the net excitation of the signal sub-elements) are available from the sub-elements and nine possible states (including the null state) are achievable given orthogonality of the signal excitations and separate bias controls. This is because the high voltage amplitude on the sine excitation is completely independent from the cosine excitation, which enables the net phase from the element (the combination of the signal sub-elements) to vary by more than just 0 degrees and 180 degrees. The excitation on the two sub-elements can be expressed as:

$$f ( t ) = {β}_{M} * {A}_{N} * sin ( 2 * π * {f}_{op} * t + {δ}_{N} ) \qquad ( 6 a )$$

$$f ( t ) = {α}_{M} * {A}_{N} * cos ( 2 * π * {f}_{op} * t + {δ}_{N} ) \qquad ( 6 b )$$

$${β}_{M} = \{ + 1 , - 1 , 0 \} \qquad ( 6 c )$$

$${α}_{M} = \{ + 1 , - 1 , 0 \} \qquad ( 6 d )$$

where β_{M} and α_{M} are the amplitudes on the high voltage lines, respectively.

The net phases that arise from simultaneous quadrature signal excitation of a sub-element divided in the signal dimension, with separate bias voltages applied to each sub-element, are illustrated in the table shown in FIG. 6C. As the table shows, the net amplitude from the element is expressed as the square root of the sum of the squares of the amplitudes on the high voltage lines.

When considering the implementation of this scheme across an entire 2D array, this approach appears to offer a significant benefit when compared to a conventional row-column array implementation capable of generating only two phases. However, the additional amplitude emanating from elements that have both the sine and cosine sub-elements biased can lead to additional energy off-axis. Indeed, as shown in FIG. 6C, in some cases, there is a square root of 2 increase in net amplitude from the element when both bias lines are applied.

This issue may be circumvented by utilizing, for example, the electrostrictive characteristics of the material or the CMUT characteristics on the membrane (e.g. bias voltage). The polarization strength in an electrostrictor is related to the bias amplitude. Eventually, the polarization strength saturates with a high enough DC bias voltage; however, at lower bias voltages the polarization strength is reduced such that the element may be shaded or apodized without affecting the element phase. Similarly, CMUTs are bias sensitive devices. The DC bias is used to provide a restoring force on the capacitive membrane, balancing the electrostatic force created when exciting the membrane with AC voltage. The DC bias can be used to control the electromechanical efficiency of the CMUT (i.e. sensitivity can be controlled with DC bias level). When the DC bias is applied, the membrane is pulled toward the bottom substrate. If the electrostatic force pulling the membrane down overcomes the restoring force of the membrane, the membrane will collapse onto the bottom substrate. This threshold voltage is called the collapse voltage. For maximum efficiency, a CMUT cell should be operated near the collapse voltage. A negative bias voltage also acts by pulling the membrane toward the bottom substrate. In either the negative bias or positive bias cases the AC excitation voltage surfs on top of the DC bias and the combination determines the polarity of the pulse produced. If a positive DC bias is applied, the combination of the bias and positive portion of the AC voltage produces a positive membrane deflection. If a negative DC bias is applied, the combination of the negative bias and the positive portion of the AC voltage will start as a net negative and create a negative deflection and a pulse with negative polarity.

For example, it is conceivable to employ a bias line with three or more different amplitude levels (e.g. resulting in a total of at least five different distinct bias levels), such as the following example bias levels that yield additional choices based on the DC voltage polarity:

$${β}_{M} = \{ + 1 , + 0.707 , - 0.707 , - 1 , 0 \} \qquad ( 7 c )$$

$${α}_{M} = \{ + 1 , + 0.707 , - 0.707 , - 1 , 0 \} \qquad ( 7 d )$$

The additional bias levels allow the net amplitude associated with the quadrature excitation of both sub-elements to be constant across the aperture for different bias aperture implementations, as shown in FIG. 6D.

A desired phase delay in the bias dimension may be calculated using the distance formula without considering the element position in the azimuth dimension. For example, in the case of a flat 2D array with 64 signal lines (azimuth) and 64 bias lines (elevation) is designed. The time delay for the elements in bias dimension is calculated using the distance formula:

$${t}_{focus} = \frac{\sqrt{{( {y}_{focus} - {y}_{element} )}^{2} + {z}_{focus}^{2}}}{{v}_{tissue}} \qquad ( 8 )$$

where t_{focus} is the time to the focus, vₜᵢₛₛᵤₑ is the velocity of sound in tissue, y_{focus} is focus position in elevation, yₑₗₑₘₑₙₜ is position of the element in the array, and z_{focus} is focus position in depth. The t_{focus} is related to the phase through the operational frequency. This relationship for a Fresnel aperture with 0 degrees and 180 degrees can be expressed as:

$${S}_{bias} = sign [ mod ( {t}_{focus} * 2 π * {f}_{p} + offset , - 2 π ) - π ] \qquad ( 9 )$$

In the present example embodiment involving the use of a set of discrete bias values that are applied to the sub-elements, the phase calculated from the distance formula may be compared to the possible discrete phases permitted with the multiple bias levels. For example, if a chosen implementation allows for five different bias levels as in FIG. 6D, then bias level assigned to a given sub-element may be determined using the lookup table shown in FIG. 6E. For example, for a desired phase of 87 degrees, FIG. 6E indicates that because 87 degrees falls between 67.5 degrees and 112.5 degrees, the amplitudes assigned to the sine and cosine sub-elements are +1 and 0 respectively.

It will be understood that the use of shading or apodization on the bias dimension may be extended beyond just three amplitudes (five bias levels) shown in FIGS. 6D and 6E. For example, an implementation may be configured to employ four different bias amplitudes (seven different bias levels) with the following available options:

$${β}_{M} = \{ + 1 , + 0.8 \; 66 , + 0.5 , - 0.5 , - 0.8 \; 66 , - 1 , 0 \} \qquad ( 10 c )$$

$${α}_{M} = \{ + 1 , + 0.866 , + 0 \text{.5} , - 0.5 , - 0.866 , - 1 , 0 \} \qquad ( 10 d )$$

This example configuration increases the number of distinct phase angles from eight to twelve, as shown in FIG. 6F. According to such implementations, the number of distinct phases in the bias (elevation) dimension is only limited by the number of possible bias levels.

Accordingly, referring now to FIGS. 6G and 6H, in some example embodiments, a row-column transducer 200 is employed to perform quadrature excitation and Fresnel focusing as follows. The row-column ultrasound transducer includes a two-dimensional array of ultrasound elements arranged along a plurality of rows 210 and columns 220, where, within each row 210, each ultrasound element 215 includes a first sub-element 216 and a second sub-element 218 residing laterally adjacent to one another. Each sub-element is capable of acoustic transduction when a bias is applied thereto, such that a phase of ultrasound waves emitted therefrom is dependent on a polarity of the bias. Each column of the two-dimensional array thus comprises a first sub-column 212 of first sub-elements and a second sub-column 214 of second sub-elements. The rows of the two-dimensional array extend along an azimuth direction and the columns of the two-dimensional array extend along the elevation direction.

The ultrasound transducer also includes a set of signal conductive paths, labeled as sine and cosine, with each signal conductive path being in electrical communication with signal electrodes of ultrasound elements residing along a respective sub-column of the two-dimensional array, such that the first sub-column and second sub-column within a given column are separately addressable by respective first and second signal conductive paths. A set of bias conductive paths, labeled HV1 and HV2, are also provided, each bias conductive path pair including a respective first bias conductive path and a respective second bias conductive path, the first bias conductive path and the second bias conductive path extending along or adjacent to a given row of the two-dimensional array, such that the first bias conductive path is configured to apply a respective bias to the first sub-elements with the given row, and such that the second bias conductive path is configured to apply a respective bias to the second sub-elements within the given row.

The ultrasound transducer array is controlled to perform a transmit operation as follows. A first bias aperture is applied to the first bias conductive paths and a second bias aperture is applied to the second bias conductive paths. The first bias aperture includes a set of first bias values and the second bias aperture includes a corresponding set of second bias values. While applying the first bias aperture and the second bias aperture, a set of transmit signals are delivered to the signal electrodes according to a time-delay aperture, such that the time-delay aperture defines transmit beamforming time delays associated with each column of the two-dimensional array, and such that for a given column having a respective beamforming time delay associated therewith, the transmit signal delivered to the first sub-column and the second sub-column have a common temporal envelope temporally delayed according to the beamforming delay, and where a waveform phase of the second sub-column is shifted, relative to that of the first sub-column, by pi/2. Accordingly, the first sub-column and the second sub-column of a given column are delivered beamformed transmit signals in quadrature.

The set of first bias values and the corresponding set of second bias values are configured such that the transmit operation results in the generation of a Fresnel aperture in the elevation direction, as explained above.

As explained below, the present method may be implemented using sub-aperture techniques, with the transmit event being implemented as a set of transmit events, each transmit event relating to a different elevation sub-aperture. The delays between the different transmit events (corresponding to the different elevation sub-apertures) being selected to reduce or eliminate the extended pulse lengths if the time delay across the aperture is greater than one wavelength, as described, for example in International Application No. PCT/CA2016/050193, titled “SYSTEMS AND METHODS OF COMBINED PHASED-ARRAY AND FRESNEL ZONE PLATE BEAMFORMING EMPLOYING DELAY-CORRECTED FRESNEL SUB-APERTURES”, which is incorporated herein by reference in its entirety.

Furthermore, as described in detail below, multiple synthetic aperture (in transmit and/or receive) may be used to address errors introduced due to the transducer bandwidth. In such implementations, a type of frequency compounding may be applied to reduce or minimize the error introduced by only using one frequency to calculate the ideal phase delay. Appropriate bias patterns are applied depending on the frequency of interest with the aperture responses of each frequency summed to give a wide bandwidth response.

The configuration shown in FIGS. 6G and 6H illustrates an example implementation in which the electrical connections for the bias conductive paths, labeled HV1 and HV2, and signal conductive paths, labeled sine and cosine, are orthogonal to each other. The figure illustrates an example implementation in which the sine excitations are routed to one edge of the transducer and the cosine excitations are routed to the opposite edge of the transducer, thereby resulting in a maximum distance between the signal traces. This enables an edge connection to be applied to the array that electrically connects between the transducer and another electrical board which may be a flexible printed circuit board (PCB, not shown). The electrical connections between the array and flexible PCB may be achieved, for example, through wire-bonding or an anisotropic conductive film or paste. The bias electrical conductive paths to the elements are formed while ensuring that they do not connect to sine and cosine sub-elements. In a non-limiting example implementation, this may be achieved, for example, by bonding the electrostrictive material to a non-conductive layer with electrical vias that are aligned to the sub-elements. It noted that the non-conductive layer may be attached to the front or back of the array and may also be used to improve the acoustic match between the array and tissue or the backing material by selecting a layer material having a suitable acoustic impedance.

In some example implementations, the conductive paths are patterned on the transducer material that connect to the bias elements, as shown, for example, in FIGS. 6I and 6J. This avoids the use of an anisotropic conductive backing and separate controls of the bias for the sine and cosine excitations. This may require a slight widening of the mechanical isolation kerf between the bias rows to allow the routing of the traces that connect the high voltage (HV) lines to the individual elements.

In some example embodiments, a square or rhombus shape for the sub-elements and arranging the sub-elements in a diamond-like pattern. FIGS. 6K and 6L show two different example implementations of two-dimensional arrays (300 and 310, respectively) with sub-elements arranged in a diamond pattern. In both cases, the element pattern enables direct connection of signal and bias conductive paths without an intervening non-conductive layer, as shown in the figures. This direct connection is possible because of the spacing between the squares or rhombuses, when arranged in the diamond pattern, creates a region that prevents the orthogonal bias or signal conductive paths from touching if the region of the element is considered conductive. In this case, the transducer is patterned on the top and bottom with the appropriate conductive paths that connect the corners of the squares or rhombuses. The connections are brought out to the transducer edges where wire bonding or anisotropic conductive film or paste is used to connect to a flexible PCB for eventual connection to a transducer electrical connector.

Accordingly, in some example implementations, a two-dimensional array of ultrasound elements is arranged in a diamond pattern defining a set of rows and columns, such that for a given transducer element, a row associated with the given transducer element extends along a first axis connecting a first pair of opposing vertices of the given transducer element, and a column associated with the given transducer element extends along a second axis connecting a second pair of opposing vertices of the given transducer element. Each ultrasound element is capable of acoustic transduction when a bias is applied thereto, such that a phase of ultrasound waves emitted therefrom is dependent on a polarity of the bias.

A set of signal conductive paths is provided such that each signal conductive path extends along a respective column of the two-dimensional array. The set of signal conductive paths include a set of odd signal conductive paths and a set of even signal conductive paths. Each even signal conductive path extends along a respective even column of the diamond pattern and each odd signal conductive paths extends along a respective odd column of the diamond pattern.

A set of bias conductive paths are also provided such that each bias conductive path extends along a respective row of the two-dimensional array. The set of bias conductive paths includes a set of odd bias conductive paths and a set of even bias conductive paths. Each even bias conductive path extends along a respective even row of the diamond pattern and each odd bias conductive path extends along a respective odd row of the diamond pattern.

The ultrasound array is controlled to perform a transmit operation as follows. A first bias aperture is applied to the set of odd bias conductive paths and a second bias aperture is applied to the set of even bias conductive paths. The first bias aperture includes a set of first bias values and the second bias aperture comprising a corresponding set of second bias values. While applying the first bias aperture and the second bias aperture, a set of transmit signals defined according to a time-delay aperture are delivered to the set of odd signal conductive paths and the set of even signal conductive paths, such that the set of transmit signals are provided to the set of even signal conductive paths in quadrature. The set of first bias values and the corresponding set of second bias values are configured such that the transmit operation results in the generation of a Fresnel aperture.

The aforementioned example embodiments were realized by subdividing or sub-dicing an element to create sub-elements that effectively represent the required phase necessary for the elevation focus, or by rotating the aperture to create a diamond pattern of elements, with each element of a traditional 2D array being effectively subdivided into four elements, with the transmit operations based on the quadrature excitation of the sets of sub-elements occurring simultaneously to engineer a composite elevation phase profile with fine phase control. Such embodiments can significantly improve the focal gain for therapy and can reduce the clutter for pulse-echo imaging. Compared to a traditional 2D row-column electrode array, these new embodiments require connections in azimuth and elevation for the sine and cosine (quadrature) excitations as well as the different bias levels. Instead of requiring N signal connections and M bias connections, 2*N signal connections and 2*M bias connections are required respectively. This is still less than a fully sampled 2D array (N*M), but quadruple the connections in a conventional row-column transducer. FIG. 6L illustrates a diamond pattern that allows the pitch of the signal lines to be different from the pitch of the bias lines.

In some example embodiments, the preceding example methods, in which quadrature (orthogonal) excitation is employed in the signal (azimuth) dimension, with separate bias apertures applied for each of the two sets of quadrature excitation signals, may be employed in a synthetic transmit-receive imaging configuration involving a sequential series of transmit and receive events, in which a Fresnel phase profile is synthetically generated by the transmit and receive operations with controllable phase resolution beyond the mere two phases that are employed in conventionally Fresnel focusing of row-column transducer arrays. As will be shown below, the use of a synthetic aperture implementation in transmit and receive enables the generation of fine phase profiles in elevation while permitting the use of only the N+M connections of a row-column electrode array.

Before describing such synthetic transmit/receive embodiments involving quadrature excitation with compounded Fresnel focusing, it is instructive to first consider the limitations of previous approaches involving the use of Fresnel focusing in row-column transducer arrays.

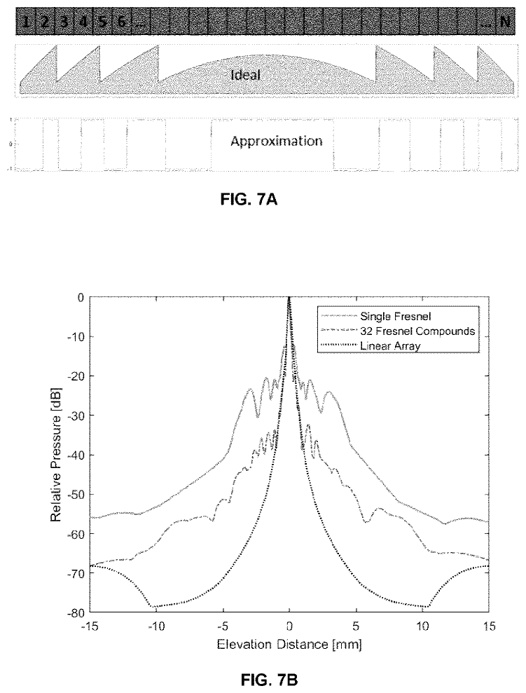

In previous work by some of the present inventors, particularly in International Patent Publication No. PCT/CA2017/051524, Fresnel lens approximations was generated by only applying varying polarity across the bias (elevation) elements at a fixed amplitude. FIG. 7A shows an example of an ideal Fresnel lens and the approximation of this lens created by changing polarity across elements. FIG. 7B shows the resulting relatively poor two-way radiation pattern from a single Fresnel approximation, simulated using Field II. The array geometry used for this simulation was a 64 element 20 MHz linear array with 1λ pitch and the cross section was simulated at f^{#}2.5. Field II simulations were carried out for a linear array geometry but with no beamforming delays inserted but the signals from each element were still summed together. To emulate the Fresnel pattern, the apodization for each element was set to either +1 or −1 corresponding to the direction of desired polarization vector (FIG. 2A).

The present example synthetic transmit/receive embodiment improves upon these prior approaches by separately transmitting pulses based on azimuth transmit signals that are in quadrature (out of phase; referred to as sine and cosine transmit signals) with different bias apertures, such that the synthetic combination of the received signals effectively results in elevation focusing according to an elevation phase profile that has a higher resolution than a conventional biphasic Fresnel aperture. This is achieved, as explained below, by controlling the bias amplitude for each elevation row individually.

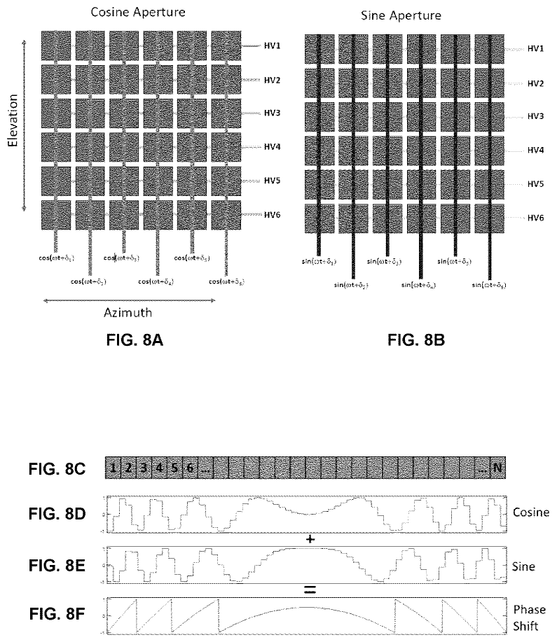

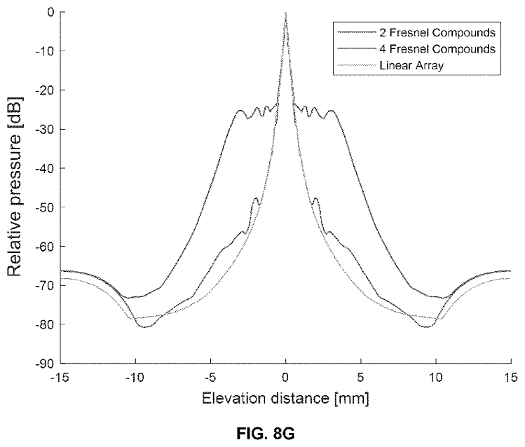

FIGS. 8A and 8B illustrate how a transmit operation involving quadrature excitation can be split into two sequential events and implemented using a conventional row-column transducer array to achieve a synthetic Fresnel lens with fine phase control. As shown in the figure, two different transmit events are performed, with the excitation signals for the two events being provided to the azimuth electrodes in quadrature. In other words, the excitation signals provided to azimuth electrodes for the second transmit event are out of phase with the excitation signals provided to the azimuth electrodes for the first transmit event. The bias apertures employed for the first and second transmit events are generated such that their synthetic combination results in a suitable approximation to a desired Fresnel phase profile. The two bias apertures are presently referred to as the “cosine” and “sine” apertures, as noted above, to denote their use with the respective quadrature excitation signals.

Mathematically, when synthetically compounding (summing) a sine and cosine wave for a given element, the resulting wave can be described as eq. 10 (in a manner similar to the description associated with equations 6a and 6b above):

$${C}_{N} cos ( 2 π ft + ϕ ) = {A}_{N} cos ( 2 π ft ) + {B}_{N} sin ( 2 π ft ) \qquad ( 10 )$$

where C_{N} and ϕ are the resulting amplitude and phase from compounding a cosine wave of amplitude A_{N} and sine wave of amplitude B_{N}. From equation 10, the following expressions can be derived for the resulting amplitude and phase of the compounded wave:

$${C}_{N} = \sqrt{{A}_{N}^{2} + {B}_{N}^{2}} \qquad ( 11 )$$

$$ϕ = {tan}^{- 1} ( \frac{- {B}_{N}}{{A}_{N}} ) \qquad ( 12 )$$

Considering equation 12, it is apparent that the net (synthetically compounded) phase of any element “N” that is pulsed sequentially with a cosine and sine wave can be controlled by simply controlling the amplitudes A_{N} and B_{N} of the two pulses. Since the transducer material is electrostrictive (or a CMUT) and can become piezoelectrically active with either positive of negative bias voltages, this provides more freedom in selecting the sine and cosine amplitudes for any given phase control. Additionally, since it may be desirable to ensure that the Fresnel lens has is constant amplitude across the transducer array, one can set C_{N}=1 as a constraint on equations 11 and 12 when generating different phase values.

FIGS. 8D and 8E show bias values (A_{N} and B_{N}, scaled to unity) of example cosine and sine bias apertures that when separately and sequentially applied to the elevation electrodes (FIG. 8C) with the respective quadrature azimuth excitation signals, result in a synthetic compounded phase profile that approximates the Fresnel phase profile shown in FIG. 8F.

Although the preceding equations can be employed to generate suitable bias apertures for use with sequential transmit operations involving azimuth signals that are in quadrature, such that the net phase associated with the synthetic combination of the two transmit events approximates a phase (delay) profile of a Fresnel lens, it was not intuitive how this approach could be adapted to a two-way radiation pattern with both transmit and receive apertures considered.

Indeed, when both transmit and receive apertures are considered, the present inventors found that the use of only two transmit events (two compounds) is insufficient to achieve performance comparable to conventional time-delayed linear array. For example, the two-way acoustic radiation pattern achieved by compounding the sine and cosine apertures for a 20 MHz, 1λ pitch, 64 element linear array aperture focused to f^{#}2.5 is shown in FIG. 8G. The figure plots the two-way radiation pattern resulting from compounding a sine and cosine aperture with TX1/RX1=sine/sine, TX2/RX2=cosine/cosine and compares the radiation pattern with that of a full linear array, shown as “2 Fresnel Compounds”. Although this combination theoretically compounds to produce a Fresnel lens with fine delay resolution, the present inventors found that it is only effective for a one-way acoustic lens.

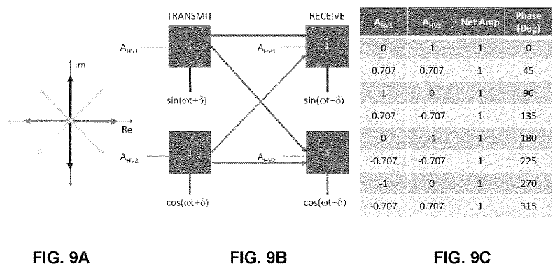

Surprisingly, the present inventors found that when at least four different transmit/receive events were synthetically compounded, a level of performance was achieved that was similar to that of a conventional time-delay focused linear array, as shown in FIG. 8G. An example implementation of a synthetic transmit/receive embodiment involving four transmit/receive events is shown in FIGS. 9A to 9C, with FIG. 9C showing example combinations of bias values that result in net phases for constructing the cosine and sine bias apertures to achieve a desired net synthetic Fresnel phase profile.

As shown in FIG. 9B, the example four-pulse transmit/receive sequence is implemented using the following bias aperture combinations: TX1/RX1=sine/sine, TX2/RX2=sine/cosine, TX3/RX3=cosine/sine, TX4/RX4=cosine/cosine (preferably, TX4/RX4 is performed prior to TX3/RX3 to limit aperture switching). The cosine aperture is employed, in transmit, with excitation signals that are in quadrature with those that are applied in transmit with the sine aperture. When the cosine aperture is employed in receive, the resulting receive signals are delayed by a time delay corresponding to a phase delay of π/2. The four transmit/receive events may be applied in any order.

The performance benefit that is gained by employing four transmit/receive events is demonstrated in FIG. 8G, which shows the radiation pattern that results from compounding a 4-pulse aperture with TX1/RX1=sine/sine, TX2/RX2=sine/cosine, TX3/RX3=cosine/sine, TX4/RX4=cosine/cosine. Remarkably, after the 4-pulse compound sequence, a radiation pattern is achieved that is very close to a theoretical fully sampled linear array radiation pattern.

The use of both the cos and sine apertures in both transmit and receive generates an effective bias (elevation) aperture with multiple phases levels, as in the preceding example embodiments. For each of the sine and the cos apertures, the bias amplitude can be calculated in the same manner as previously described in the preceding example sub-diced/subdivided quadrature transmit embodiments.

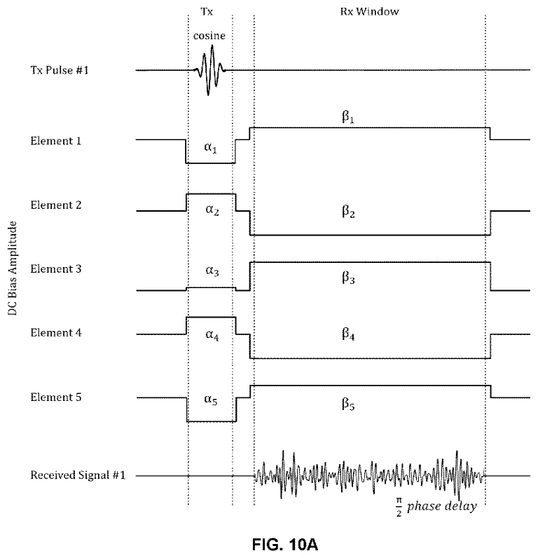

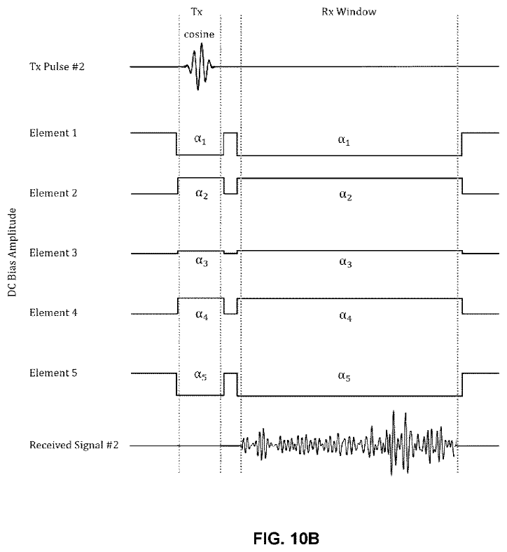

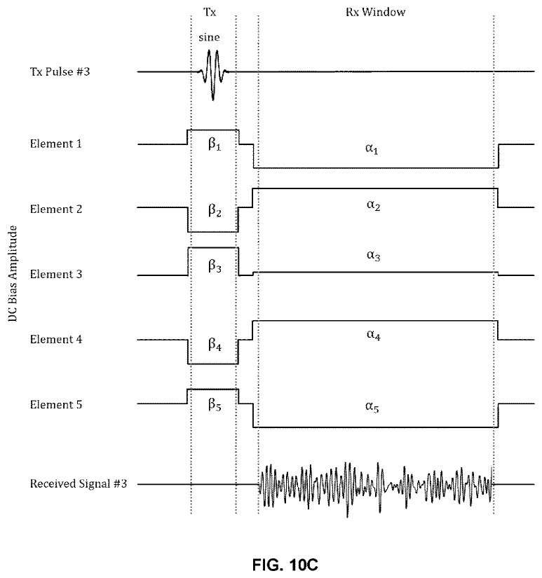

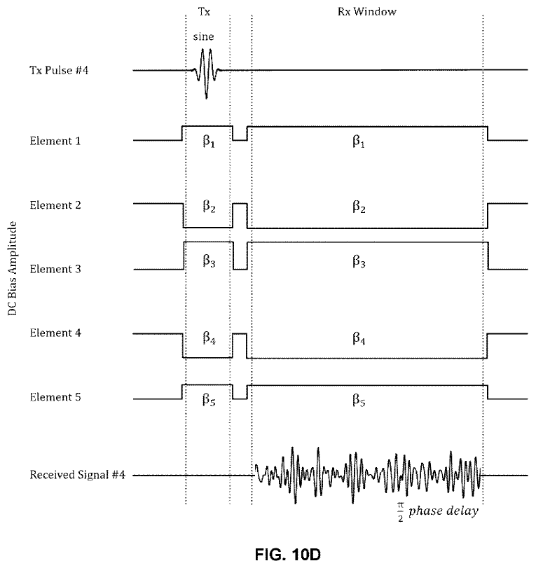

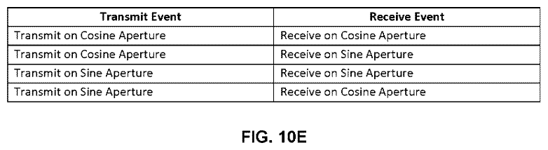

FIGS. 10A-10D illustrate the transmit pulse sequence for an example embodiment having 5 bias electrodes (5 rows of elements in the elevation direction). Referring first to FIG. 10A, a cosine/sine transmit event is illustrated. The first row of the figure shows the timing of the transmit pulse delivered to the azimuth electrodes. This transmit signal set is referred to as a “cosine” transmit signal and is delivered while applying the cosine bias aperture to the elevation electrodes, as shown in the figure by the α₁ to α₅ bias levels applied to the elevation electrodes (while the figure only shows a single transmit signal, it will be understood that a set of transmit signals are delivered to the set of azimuth electrodes). The received signal is shown at the bottom of the figure and is referred to as a “sine” signal and is received while applying the sine bias aperture to the elevation electrodes, as shown in the figure by the β₁ to β₅ bias levels applied to the elevation electrodes (while the figure only shows a single received signal, it will be understood that a set of received signals are received from the set of azimuth electrodes). The received sine signal is delayed by a time delay corresponding to a phase shift of π/2, so that the sine signal is detected in quadrature with the cosine signals that are received without delay, as described in further detail below.

FIG. 10B shows a cosine/cosine transmit event, where the first row of the figure shows the timing of the cosine transmit pulse delivered to the azimuth electrodes while applying the cosine bias aperture to the elevation electrodes, as shown in the figure by the α₁ to α₅ bias levels applied to the elevation electrodes. The received signal is shown at the bottom of the figure and is referred to as a “cosine” signal and is received while applying the cosine bias aperture to the elevation electrodes, as shown in the figure by the α₁ to α₅ bias levels applied to the elevation electrodes. Unlike the received sine signal in FIG. 10A, the received cosine signal is not delayed, so that it is quadrature with the received sine signals detected in other transmit/receive events.

FIG. 10C shows a sine/cosine transmit event, where the first row of the figure shows the timing of the sine transmit pulse delivered to the azimuth electrodes while applying the sine bias aperture to the elevation electrodes, as shown in the figure by the β₁ to β₅ bias levels applied to the elevation electrodes. As can be seen by comparing FIG. 10C to FIG. 10B, the sine transmit pulse is generated in quadrature with respect to the cosine transmit pulse. The received cosine signal is shown at the bottom of the figure and is received while applying the cosine bias aperture to the elevation electrodes, as shown in the figure by the α₁ to α₅ bias levels applied to the elevation electrodes.

FIG. 10D shows a sine/sine transmit event, where the first row of the figure shows the timing of the sine signal pulse delivered to the azimuth electrodes while applying the sine bias aperture to the elevation electrodes, as shown in the figure by the β₁ to β₅ bias levels applied to the elevation electrodes. The sine transmit pulse is generated in quadrature with respect to the cosine transmit pulse of the other transmit/receive events. The received sine signal is shown at the bottom of the figure and is received while applying the sine bias aperture to the elevation electrodes, as shown in the figure by the β₁ to β₅ bias levels applied to the elevation electrodes.

The four sets of received signals that result from the four pulse-echo events are summed (synthetically compounded), with the application of the π/2 phase shift when the sine aperture is employed in receive, to achieve receive signals that correspond to an elevation focus associated with the net phase profile of the bias apertures that approximate a Fresnel lens.

It is noted that addition to minimizing the additional number of channels employed, when compared to the previous example embodiments involving sub-diced/subdivided elements, the present example synthetic aperture embodiments improve the transmit sensitivity and received SNR since more active material is used in the sine and cosine transmit apertures and multiple received signals are averaged together, which reduces the overall noise level.

While the four transmit/receive events may be performed in any order, it may be beneficial to employ a sequence order shown that minimizes the number of switches between the cosine and sine apertures, since significant switching may cause heat generation as the elements are biased to a different voltage. A non-limiting example of such a sequence is shown in FIG. 10E.

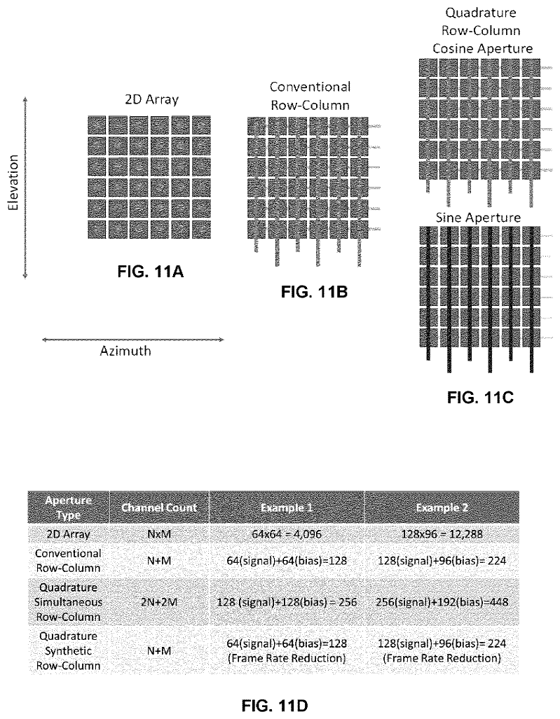

FIGS. 11A, 11B and 11C contrast the configurations of a conventional fully sampled 2D array and a conventional row-column array with the present synthetic quadrature transmit/receive Fresnel focusing embodiments. The 2D array shown in FIG. 11A is fully sampled and is the gold standard when considering the beam performance at the focus. The conventional row-column transducer, shown in FIG. 11B, reduces the number of electrical connections. Unfortunately, it significantly underperforms the 2D array in terms of performance for both ultrasound therapy and imaging because it is weakly focused. The synthetic quadrature transmit/receive Fresnel focusing approach, shown in FIG. 11D, circumvents this issue by offering a wider range of possible phases in elevation to focus the beam. As noted above, the quadrature excitation methods of the present disclosure can be implemented by sub-dicing/subdividing the elements and operating the cosine and sine apertures simultaneously, or by employing synthetic transmit and synthetic receive imaging. The table in FIG. 11D summarizes the channel count for method, including both the sub-diced/subdivided quadrature implementation and the synthetic quadrature transmit/receive implementation. As can be seen in the figure, when compared to a conventional fully-sampled array, the total number of channels decreases by one to two orders of magnitude for the two quadrature methods.

The example synthetic transmit/receive embodiments described above employ at least four transmit/receive events in order to utilize all combinations of two sine and cosine apertures in transmit and receive, where the sine and cosine apertures are selected such that a net synthetic aperture associated therewith generates a phase profile, in the elevation direction, that approximates a Fresnel lens.

In some example implementations, the quadrature signals provided to the azimuth electrodes may be time-delay beamformed to focus along a selected image line. In such cases, the signals provided to the azimuth electrodes maintain the same focused azimuthal image line among at least one set of four synthetic transmit/receive operations.

However, in other example implementations that involve insonification of a wide area and employ azimuthal compounding, such as plane wave or diverging wave compounded azimuth imaging, the quadrature signals provided to the azimuth electrodes may vary among the four synthetic transmit/receive events. In such cases, a “sine” set of signals employed during one transmit operation may correspond to one plane wave or diverging wave insonification, and a set of “cosine” signals employed during another transmit operation may correspond to a different plane wave or diverging wave insonification that is shifted, by a phase difference of π/2, relative to the excitation waveform underlying the “sine” set of signals.

Although the example implementations described above employ the same pair of sine and cosine bias apertures in transmit and receive, other example implementations may employ a different pair of bias apertures in transmit and receive, such that a transmit pair of sine and cosine apertures are configured to generate a synthetic Fresnel focus at a first elevation location and a receive pair of sine and cosine apertures are configured to generate a synthetic Fresnel focus at a second elevation location that is different from the first elevation location. Such use of two different elevation foci in the elevation dimension may be beneficial in improving the slice thickness. For example, the elevation focus may be at a depth of 10 mm in transmit and at a depth of 15 mm in receive.

In some example embodiments of the present synthetic transmit/receive quadrature methods, a combination of azimuth and Fresnel compounding may be employed. In International Patent Publication No. PCT/CA2017/051524, titled “SYSTEMS AND METHODS FOR ULTRASOUND BEAMFORMING USING COHERENTLY COMPOUNDED FRESNEL FOCUSING”, which is incorporated herein by reference in its entirety, some of the present inventors had demonstrated a new technique of simultaneous two-way focusing named simultaneous azimuth and Fresnel elevation (SAFE) compounding. 3D images were generated using an electrostrictive crossed electrode (RCA), where one set of electrodes is employed to perform a standard ultrafast imaging approach, such as plane/diverging wave imaging, while the orthogonal set of electrodes acts as a reconfigurable elevation lens.

According to such methods, the plane/diverging wave imaging provides a focused 2D image in the azimuth plane using a plurality of transmit pulses, while the slice (elevation) resolution is accomplished using an approximation of a Fresnel lens controlled with DC bias voltages on an electrostrictive substrate or CMUT. Not only does this produce a two-way focused beam in both dimensions, but also removes the need to switch RF channels and ground references reducing system complexity. In this previous study, biphasic approximation to a Fresnel lens was employed by pulsing the elements with either a positive or negative polarity of fixed amplitude to create the focused elevation beam. However, it was demonstrated that the approximate Fresnel pattern could be changed from pulse-to-pulse simultaneous to incrementing each plane/diverging wave angle, without affecting the azimuth image. The advantage to this was that simultaneously compounding different elevation Fresnel patterns improved the elevation beam profile, axial resolution, and overall image quality. Despite elevation beam improvements with compounding, the elevation secondary lobes were still not suppressed enough to achieve equivalent image contrast to the azimuth dimension.

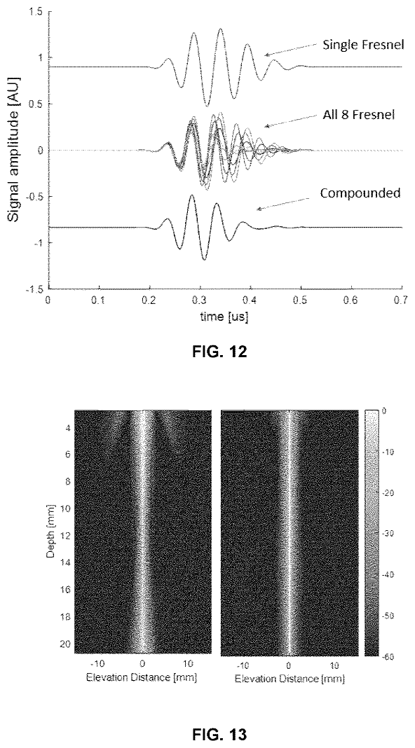

In the previous study on SAFE compounding, it was demonstrated that compounding different Fresnel patterns that are focused to nearly the same focal location but in different enough location to change the Fresnel pattern, the resulting axial pulse length is reduced. This is because the first part of each pulse is in phase and results in constructive interference, whereas the tail region of the pulses are out of phase and compound destructively. FIG. 12 shows an example of pulses resulting from 8 slightly different focal locations and the resulting compounded (normalized) pulse. By compounding these 8 pulses the bandwidth increases from approximately 35% to 50%. Another method for increasing pulse bandwidth is by activating only part of the Fresnel aperture and then on subsequent pulses activating the remainder of the Fresnel aperture but group delaying the sub-apertures such that they compensate for the temporal delay between sub-apertures. The drawback to this approach is that the frame rate is reduced in proportion to the number of sub-apertures.

These methods may be employed to further adapt the present example synthetic transmit/receive quadrature excitation Fresnel focusing methods. For example, the pulse bandwidth can be increased through compounding sequential Fresnel patterns that have slightly different focal locations in combination with compounding diverging waves in the axial direction.

As described above, four transmit pulses are needed to achieve a synthetic (composite) Fresnel phase pattern with fine phase delay resolution. Therefore, when performing diverging or plane wave compounding in the azimuth direction over a set of M azimuthal compounds that include at least 8 transmit pulses, the elevation focal location of the synthetic Fresnel lens may be modified among at least two different groups of four transmit pulses.

Such an example implementation involves simultaneously compounding the elevation aperture to the azimuth, and therefore when compounding over multiple elevation focal locations (each elevation focal location requiring at least four transmit/receive events) it is preferable to not exceed the number of compounds needed to generate a high-quality azimuth image. For example, the present inventors have found that for most diverging wave approaches, a relatively high-quality image can be generated with 32 compounds, and therefore it is desirable not to exceed 32 total compounds in either dimension. Such a constraint permits the focal location to be moved and compounded among 8 separate fine resolution Fresnel patterns (8*4=32).

FIG. 13 shows a full radiation pattern simulated for the 20 MHz 64-element 1λ pitch 1D array over 32 total compounds using this example approach. The radiation pattern was simulated over a range of depths between 3 mm and 20 mm. This radiation pattern was compared to a dynamically beamformed linear array with identical array geometry. The radiation pattern from the Fresnel compounded array is nearly equivalent to that of the ideally beamformed linear array. In the example section below, this example embodiment is implemented as a RCA that simultaneously focuses the beam in both azimuth and elevation through compounding.

Another example compounding method employs the use of sub apertures, reducing the number of operational elements during each of the set of four transmit/receive events. For example, a sliding sub-aperture can be employed during the four transmit/receive events, shifting the aperture (e.g. by one element) among the transmit/receive events. Such an approach can result in an improvement in the secondary lobes of the radiation pattern.

In some example implementations, two or more compounding strategies may be combined. For example, a combination of sub-aperture compounding (compounding with each set of four synthetic transmit/receive operations and Fresnel focus compounding (compounding among multiple sets of four transmit/receive operations) may be employed to improve the system performance.

Accordingly, in some example embodiments, a synthetic transmit/receive quadrature excitation Fresnel focusing method is provided that employs a row-column ultrasound transducer. The row-column ultrasound transducer includes a two-dimensional array of ultrasound elements arranged along a plurality of rows and columns, each ultrasound element being capable of acoustic transduction when a bias is applied thereto, such that a phase of ultrasound waves emitted therefrom is dependent on a polarity of the bias. The row-column ultrasound transducer includes a set of signal electrodes, each signal electrode extending along a respective column of the two-dimensional array, and a set of bias electrodes, each bias electrode extending along a respective row of the two-dimensional array. The row-column transducer array is controlled to perform a set of at least four synthetic transmit and receive operations.

A first transmit operation and first receive operation are performed by (i) applying a first transmit bias aperture to the bias electrodes while delivering a first set of transmit signals to the signal electrodes, and (ii) applying a first receive bias aperture to the bias electrodes while receiving a first set of receive signals from the signal electrodes.

A second transmit operation and second receive operation are performed by (i) applying the first transmit bias aperture to the bias electrodes while delivering a second set of transmit signals to the signal electrodes, where the second set of transmit signals are generated according to a driving waveform that is in phase with a corresponding driving waveform employed to generate the first set of transmit signals, and (ii) applying a second receive bias aperture to the bias electrodes while receiving a second set of receive signals from the signal electrodes, and applying a time delay to the second set of received signals, the time delay corresponding to a phase delay of pi/2.

A third transmit operation and third receive operation are performed by (i) applying a second transmit bias aperture to the bias electrodes while delivering a third set of transmit signals to the signal electrodes, the third set of transmit signals being generated according to a driving waveform that is phase shifted, relative to a corresponding driving waveform employed to generate the first set of transmit signals, by pi/2, and (ii) applying the first receive bias aperture to the bias electrodes while receiving a third set of receive signals from the signal electrodes.

A fourth transmit operation and fourth receive operation are performed by (i) applying the second transmit bias aperture to the bias electrodes while delivering a fourth set of transmit signals to said signal electrodes, the fourth set of transmit signals being generated according to a driving waveform that is phase shifted, relative to a corresponding driving waveform employed to generate the first set of transmit signals, by pi/2, and (ii) applying the second receive bias aperture to the bias electrodes while receiving a fourth set of receive signals from the signal electrodes, and applying a time delay to the fourth set of received signals, the time delay corresponding to a phase delay of pi/2.

The first, second, third and fourth sets of receive signals are summed to obtain a summed set of receive signals and the summed set of receive signals are beamformed in the azimuth dimension to generate an image.

The first set of transmit signals, the second set of transmit signals, the third set of transmit signals and the fourth set of transmit signals are selected such that the resulting plurality of ultrasound pulses generated therefrom spatially overlap with each other in a far field region, where the first transmit bias aperture and the second transmit bias aperture are configured to generate a synthetic transmit Fresnel aperture when the set of synthetic transmit and receive operations are compounded; and where the first receive bias aperture and the second receive bias aperture are configured to generate a synthetic receive Fresnel aperture when the set of synthetic transmit and receive operations are compounded.

As noted above, the synthetic transmit Fresnel aperture and the synthetic receive Fresnel aperture may be configured to generate common or different elevation foci. Moreover, the bias apertures may be configured such that an amplitude associated with the synthetic transmit Fresnel aperture and/or is equal for at least two rows of the synthetic transmit Fresnel aperture, and/or such that an amplitude associated with the synthetic transmit Fresnel receive and/or is equal for at least two rows of the synthetic receive Fresnel aperture.

The bias levels of the first transmit bias aperture and the bias levels of the second transmit bias aperture may be obtained from a lookup table, the lookup table associating, for each phase range of a plurality of phase ranges, a suitable first transmit aperture bias value selected from the discrete set of bias levels and a suitable second transmit aperture bias level selected from the discrete set of bias levels, such that the synthetic transmit Fresnel aperture generated by the compounding of the transmit and receive operations approximates a desired transmit Fresnel aperture.

Likewise, the bias levels of the first receive bias aperture and the bias levels of the second receive bias aperture may be obtained from a lookup table, the lookup table associating, for each phase range of a plurality of phase ranges, a suitable first receive aperture bias value selected from the discrete set of bias levels and a suitable second receive aperture bias level selected from the discrete set of bias levels, such that the synthetic receive Fresnel aperture generated by the compounding of the transmit and receive operations approximates a desired ideal receive transmit Fresnel aperture.

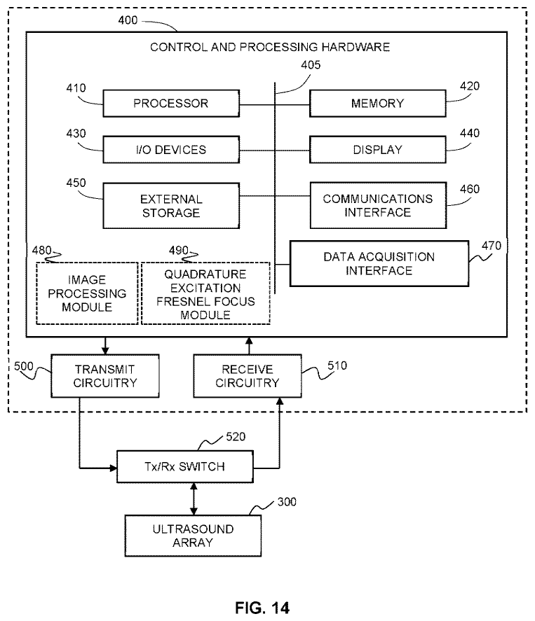

Referring now to FIG. 14, an example imaging system is illustrated for performing quadrature excitation Fresnel focusing with an ultrasound array. The example system includes an ultrasound array 300 that includes a set of ultrasound transducer array elements (e.g. piezoelectric elements, which may be a component of an ultrasound imaging device, such as an ultrasound imaging endoscope), transmit circuitry 500 for delivering transmit voltage pulses to the ultrasound array 300, a transmitter-receiver switch 520, receive circuitry 510 for detecting receive signals from the ultrasound array 300, and control and processing hardware 200 (e.g. a controller, computer, or other computing system). The transmitter-receiver switch 520 and receive circuitry 510 are employed for imaging implementations but may be absent in transmit-only implementations, for example, in some therapeutic applications.

Control and processing hardware 200 is employed to control transmit circuitry 300 and Tx/Rx switch 520, and for processing the receive signals obtained from receive circuitry 510. As shown in FIG. 14, in one embodiment, control and processing hardware 300 may include a processor 410, a memory 420, a system bus 405, one or more input/output devices 430, and a plurality of optional additional devices such as communications interface 460, display 440, external storage 450, and data acquisition interface 470.

The present example methods of performing quadrature excitation Fresnel focusing via a row-column transducer array can be implemented via processor 410 and/or memory 420. As shown in FIG. 14, the control of the delivery of quadrature excitation transmit signals to the azimuth electrodes, the application of suitable bias apertures to the elevation electrodes in transmit and receive, and beamforming of receive signals may be implemented by control and processing hardware 400, via executable instructions represented as quadrature excitation Fresnel focus module 490. The control and processing hardware 400 may include and execute scan conversion software (e.g. real-time scan conversion software) or other image processing functionality as represented by image processing module 480.

The functionalities described herein can be partially implemented via hardware logic in processor 410 and partially using the instructions stored in memory 420. Some embodiments may be implemented using processor 410 without additional instructions stored in memory 420. Some embodiments are implemented using the instructions stored in memory 420 for execution by one or more general purpose microprocessors. In some example embodiments, customized processors, such as application specific integrated circuits (ASIC) or field programmable gate array (FPGA), may be employed. Thus, the disclosure is not limited to a specific configuration of hardware and/or software.

Referring again to FIG. 14, it is to be understood that the example system shown in the figure is not intended to be limited to the components that may be employed in a given implementation. For example, the system may include one or more additional processors. Furthermore, one or more components of control and processing hardware 400 may be provided as an external component that is interfaced to a processing device. For example, as shown in the figure, any one or more of transmit circuitry 500, receive circuitry 510, and Tx/Rx switch 520 may be included as a component of control and processing hardware 400 (as shown within the dashed line), or may be provided as one or more external devices.

While some embodiments can be implemented in fully functioning computers and computer systems, various embodiments are capable of being distributed as a computing product in a variety of forms and are capable of being applied regardless of the particular type of machine or computer readable media used to actually effect the distribution.

At least some aspects disclosed herein can be embodied, at least in part, in software. That is, the techniques may be carried out in a computer system or other data processing system in response to its processor, such as a microprocessor, executing sequences of instructions contained in a memory, such as ROM, volatile RAM, non-volatile memory, cache or a remote storage device.

A computer readable storage medium can be used to store software and data which when executed by a data processing system causes the system to perform various methods. The executable software and data may be stored in various places including for example ROM, volatile RAM, nonvolatile memory and/or cache. Portions of this software and/or data may be stored in any one of these storage devices. As used herein, the phrases “computer readable material” and “computer readable storage medium” refers to all computer-readable media, except for a transitory propagating signal per se.

### EXAMPLES

The following examples are presented to enable those skilled in the art to understand and to practice embodiments of the present disclosure. They should not be considered as a limitation on the scope of the disclosure, but merely as being illustrative and representative thereof.

### Example 1: Simulation Methods for Therapy

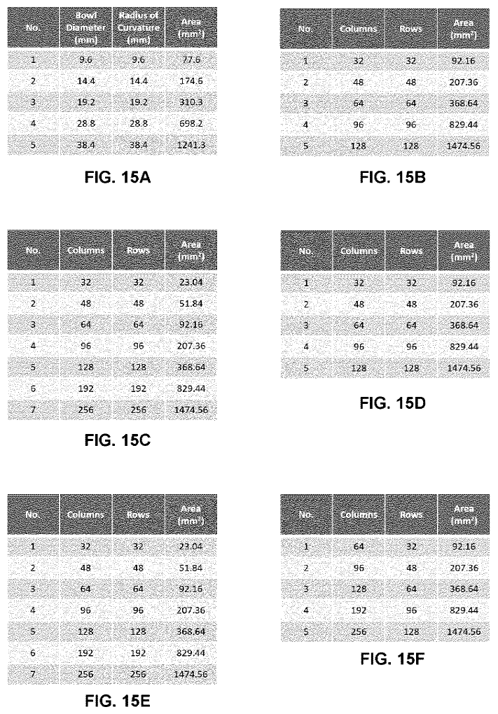

The benefits of the quadrature aperture were assessed using Field II and different aperture sizes for an operational frequency of 5 MHz. Focused bowls, 2D arrays at wavelength pitch and half-wavelength pitch, row-column transducers at wavelength pitch and half-wavelength pitch, and quadrature apertures at half-wavelength pitch (signal) and wavelength pitch (elevation) were simulated to measure focal gain. FIGS. 15A to 15E list the attributes of each aperture simulated. The f # of the aperture was one in all cases.

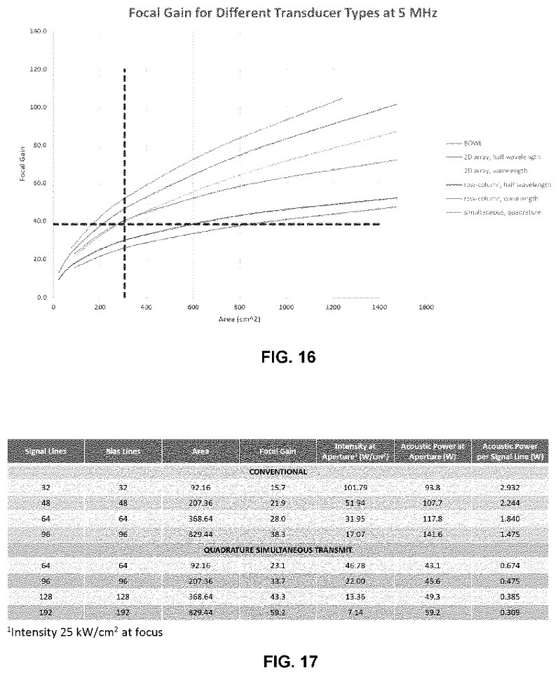

FIG. 16 shows the focal gain results for the six different apertures previously described. As expected, the focused bowl is the gold standard for ultrasound therapy. This is followed by the 2D array half-wavelength pitch and wavelength pitch respectively. At the other end are the conventional row-column transducers with the wavelength pitch generating the worst results. The quadrature row-column transducer performs nearly as well as a 2D array with a wavelength pitch. This can have significant benefits to both performance and risk mitigations. For a device area of 300 cm², the bowl has a focal gain of approximately 50 whereas the row-column transducer has a focal gain of about 30. Since intensity is related to the focal gain squared, a difference of only 20 is magnified. For example, the bowl generates an intensity that is approximately 3 times greater than the conventional row-column transducer at a wavelength pitch. This difference is significantly reduced when the quadrature aperture is applied with a focal gain of approximately 40. This focal gain is approximately identical to the focal gain obtained with the 2D array at a wavelength pitch.

FIG. 17 summarizes the differences between the conventional row-column transducer configurations and the quadrature row-column configurations. This table shows that although the number of signal and bias lines increase, the focal gain shows dramatic improvement (also see FIG. 16). In addition, the amount of aperture power necessary to achieve 25 kW/cm² at the focus decreases by over a factor of two. The power for each signal line is reduced by over a factor of four. This improved efficiency minimizes transducer heating during therapy delivery and reduces the power requirements per channel which gives additional design flexibility for the transmit electronics.

### Example 2: Simulated Array for Synthetic Transmit/Receive Quadrature Method

A 30 MHz, 1D phased array was simulated in Field II to demonstrate the bias-controlled lens. The array was given 64 elements with wavelength pitch. The RF signals from all of the elements were summed to model having only one signal channel (i.e. equivalent to applying the biases to the rows and having signal channels along a column of a crossed electrode array).

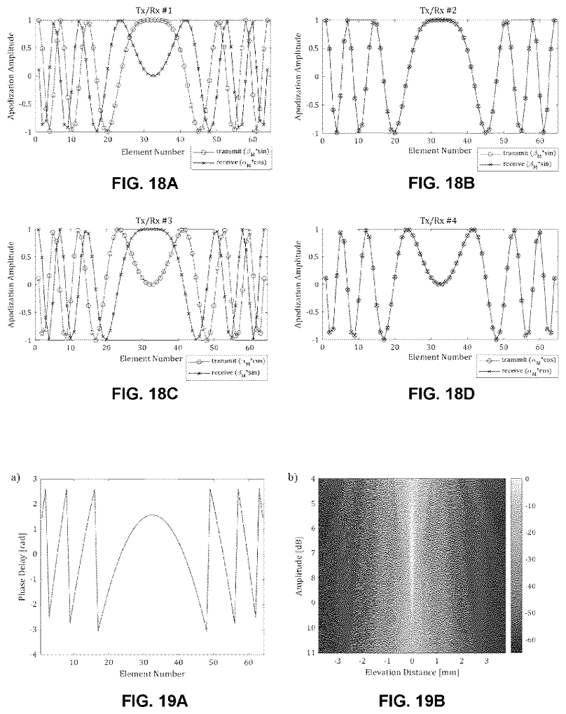

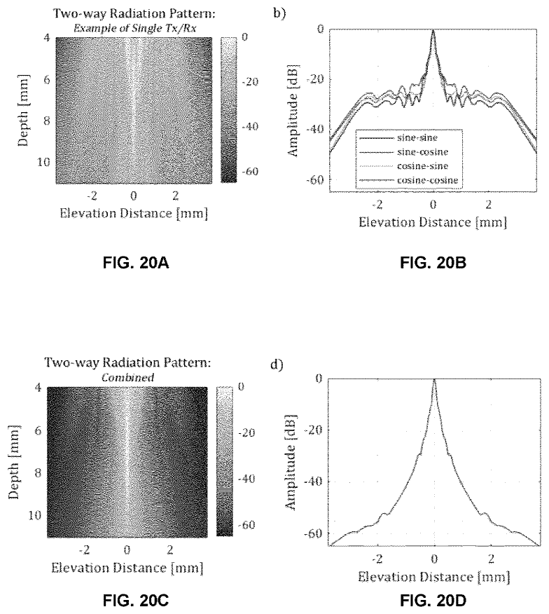

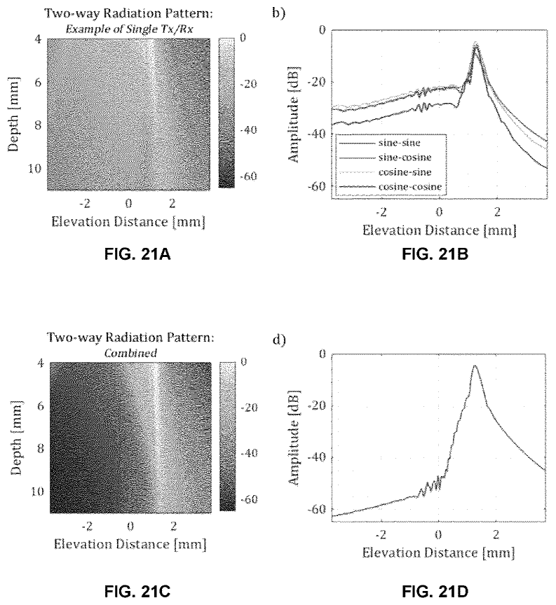

The biasing amplitude was modelled using apodization weighting for each transducer element. An example set of bias amplitudes, α_{M} and β_{M}, are shown in FIGS. 18A-18D for the four requisite transmit/receive events. The results from the bias amplitude-controlled focus will be compared to a case where the exact phase delays are applied to the array. FIG. 19A shows the exact phase delays required for a focus in front of the array while FIG. 19B shows the two-way radiation pattern created by the phase delayed aperture. FIGS. 20A-20D and FIGS. 21A-21D show two-way radiation pattern results for the bias-controlled quadrature aperture demonstrating both an on-axis focus and steering to 10 degrees. The results from the combined apertures on-axis shown in FIG. 20C is comparable to the result from the array with exact phase delays applied. Combining the bias-controlled orthogonal apertures reduced the secondary lobe level by greater than 20 dB. The cost of the improved focus is only the four transmit/receive events required to achieve the synthetic quadrature apertures.

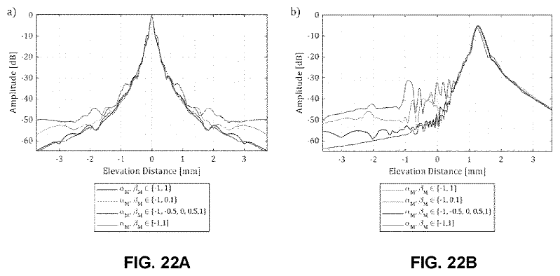

A discrete set of bias amplitudes also provides a good quality focus when combined with the synthetic quadrature apertures. As a consequence of using a discrete set of elements, the ideal amplitude curve is sampled at a coarse spatial interval. FIG. 22 shows the two-way radiation patterns for different bias amplitude constraints, 2 levels ({−1.0, 1.0}), 3 levels ({−1.0, 0, 1.0}), 5 levels ({−1.0, −0.5, 0, 0.5, 1.0}) or continuous bias levels.

As previously described, there are compounding techniques that can be employed to improve the bias-controlled focusing. In two techniques described below multiple biasing patterns are repeated for the four pulses needed to achieve the synthetic orthogonal aperture. The first technique shifts the Fresnel lens to various points around the main focal location, largely differing in the elevation position and keeping the axial depth the same. This technique shows improvement in the radiation pattern in the width of the main beam. Such an implementation is described in the example below.

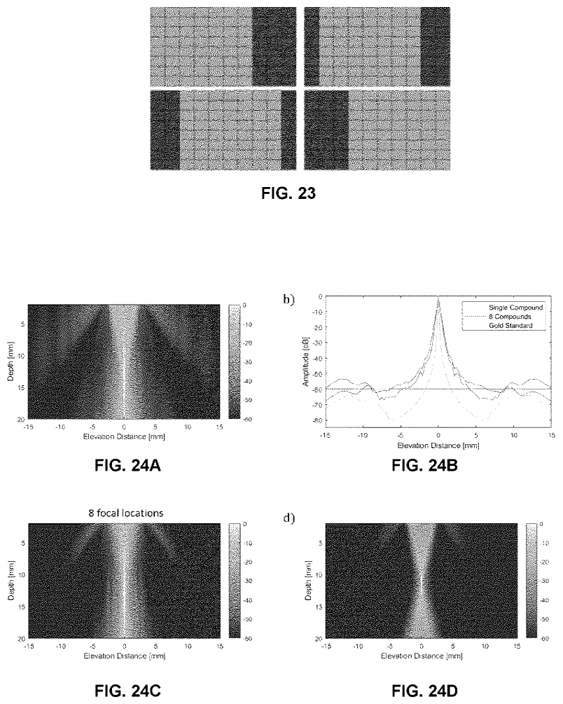

A second compounding technique uses sub apertures, reducing the number of operational elements and shifting the aperture by one element for each of the set of four transmit/receive events. This results in an improvement in the secondary lobes of the radiation pattern. FIG. 23 shows an example illustrative implementation employing an 8×10 array, where the dark elements are inactivated with an apodization level of zero. The active elements shift by one element per Fresnel scheme in this case. For demonstration, there are four shifts in total. This reduction in the number of elements used slightly decreases the area of the operational aperture, shortening the pulse in the time domain.

The two compounding strategies described above can be combined to tune results for a specific application.

A 20 MHz, 2D array was simulated in Field II to demonstrate the compounding improvement for the bias-controlled elevation lens. The simulated array was 64 elements in azimuth and 60 elements in elevation with 72 μm and 88 μm pitch respectively. The RF signals along the elevation dimension were summed to simulate a crossed electrode array. Each simulation employed 8 compounds that were repeated 4 times to create the synthetic quadrature (orthogonal) aperture for a total of 32 transmit/receive events. All the radiation patterns were compared to a gold standard case where the theoretically perfect delay profile is applied (not phase wrapped).

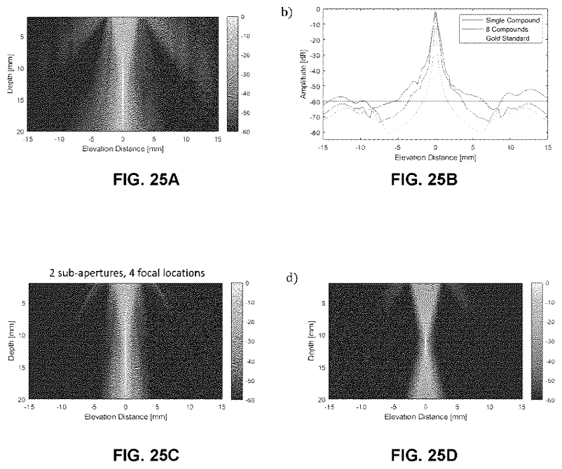

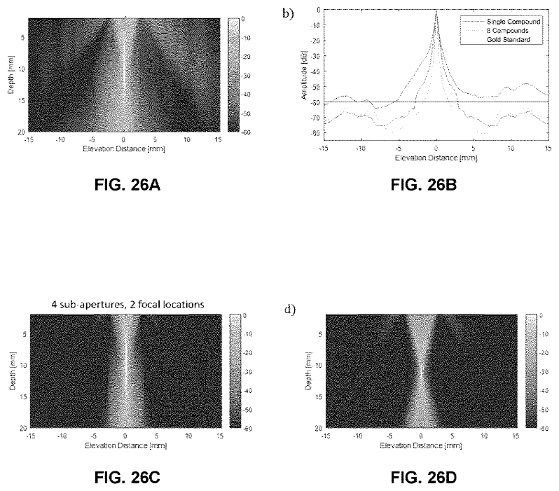

FIGS. 24A-24D, 25A-25D and 26A-26D show simulated radiation patterns for different combinations of the two compounding approaches (multiple Fresnel patterns created by moving the focal location and sliding a large sub-aperture). FIGS. 24A-24D show the results for a simulation in which 8 focal locations were compounded without employing a sliding sub-aperture. FIGS. 25A-25D show the results for a simulation in which 4 focal locations were compounded while employing two sub-apertures. FIGS. 26A-26D show the results for a simulation in which 2 focal locations were compounded while employing four sub-apertures.

### Example 3: Simulated Array for Synthetic Transmit/Receive Quadrature Method with Compounded Azimuth Diverging Wave Imaging

A 20 MHz 64 by 64 element RCA was designed by simulating beam profiles in three dimensions using Field II. The radiation patterns were simulated by implementing a full 64x64 element 2D matrix simulation in field II, however, an apodization function was applied across the elevation dimension with values ranging between −1 and 1 to emulate the effect of bias voltage applied to the elevation elements. To emulate a row-column array, The RF signals from the signal elements of the matrix array were summed across the elevation aperture to model having only one signal channel per column electrode (i.e. equivalent to applying the biases to the rows and having signal channels along each a column of a crossed electrode array). For a given tx/rx event, no beamforming was performed in the elevation dimension relying solely on the apodization pattern, compounding and the π/2 group delays for beam focusing in the elevation plane.

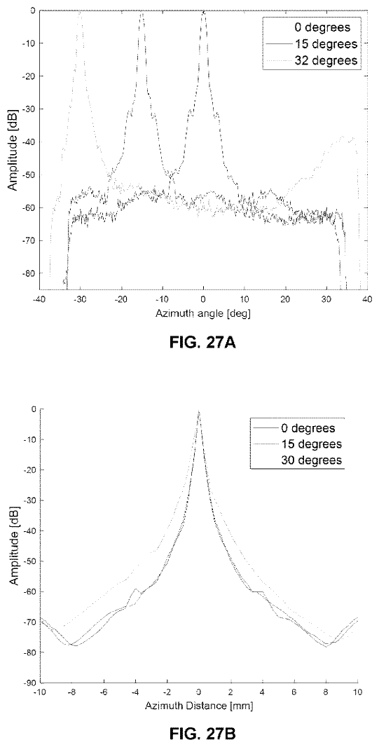

In the azimuth plane, diverging wave imaging was implemented based on the method described in International Application No. PCT/CA2016/050193. Since the azimuth plane requires beam steering but the elevation plane does not (for a 0-degree lens), different element pitches were selected for the two planes of 54 and 80 microns respectively. This allowed for the maximization of resolution and minimization of grating lobe artifacts in each dimension. FIG. 27A shows simulated beam profiles for the diverging wave dimension at steering angles of 0, 15, and 30 degrees for 32 diverging wave compounds (cross section of the radiation pattern at a depth of 11 mm). FIG. 27B shows the radiation pattern simulated for the elevation dimension at a zero-degree steering angle only. The elevation pattern was simulated for when the azimuth beam was steered to 0, 15, and 30 degrees and this resulted in relatively little difference in the beam profile, demonstrating how independent the two dimensions are of each other. Since it is possible to compound up to 32 bias patterns simultaneous to the diverging wave image generation without affecting frame rate, FIG. 27B shows the radiation pattern resulting from 8 different groups of 4 optimized Fresnel lenses that were all compounded together to bring the total number of pulses to 32. The 8 different Fresnel patterns were selected such that they were focused to slightly different focal locations, the focal locations being shifted such that the Fresnel pattern is slightly changed, resulting in improved axial resolution.

### Example 4: Bias Hardware Design

A 64-channel dynamic biasing system was developed and a block diagram of the major components are shown in FIG. 7. An FPGA board was developed and connected to 8 parallel daughter cards that convert digital data from the FPGA to analog DC voltages ranging from +/−50V. 8-bit parallel digital to analog chips are used to convert the data from the FPGA to an analog signal, and this is passed through to a high voltage circuit based on simple high-voltage op-amps.

### Example 5: Array Fabrication

An example array was fabricated in a very similar way to that described in Latham et al. [Latham, C. Samson, J. Woodacre, and J. Brown, “A 30 MHz, 3D Imaging, Forward Looking Miniature Endoscope Based on a 128-Element Relaxor Array,” IEEE Trans. Ultrason. Ferroelectr. Freq. Control, 2020, doi: DOI 10.1109/TUFFC.2020.3027907.]. First a 1-3 composite was created from a monolithic high density electrostrictive ceramic. A crossed electrode pattern was then fabricated on the top and bottom of the substrate and then wire bonded to circuit board interposers that connect to the cable and system. The primary difference between the presently fabricated array and the previously fabricated array is that there is a different element pitch between the top and bottom set of electrodes. This was because one set of electrodes was tailored towards phased array beam steering and one set was tailored towards a linear array aperture multiplexing approach with only zero-degree focused beams. A single quarter wavelength matching layer was deposited through vacuum deposition.

### Example 6: Array Characterization

The array performance was characterized through electrical impedance measurements, pulse echo bandwidth measurements, sensitivity vs. DC bias voltage curves, and acoustic beam radiation patterns. Electrical impedance measurements were made using an RF impedance analyzer under a 40-volt bias. Pulse echo measurements were measured by pulsing a single element of the array and measuring the echo off of a quartz reflector at a depth of 6 mm.

### Example 7: Theory of Phase Delay Control in Synthetic Transmit/Receive Quadrature Excitation Fresnel Focusing Using Four Transmit/Receive Events

A discrete set of bias levels can be employed with synthetic quadrature (orthogonal) transmit and receive apertures to achieve a discrete set of element-to-element phase delays. If the bias amplitude is not limited to a set of discrete values, exact phasing can be configured for each element in an aperture (within a wavelength) to mimic a Fresnel lens. The effective phase of each element by choosing the appropriate scaled linear combination of a sine and cosine aperture. As noted above, the result of combining the scaled orthogonal excitations becomes,

$${α}_{M} * cos ( 2 * π * {f}_{op} * t ) + {β}_{M} * sin ( 2 * π * {f}_{op} * t ) = {c}_{M} * cos ( 2 * π * {f}_{op} * t + φ ) \qquad ( 13 )$$

where the bias amplitudes, α_{M} and β_{M}, are chosen to tune the phase delay, φ, with,

$$φ = {tan}^{- 1} \frac{- {β}_{M}}{{α}_{M}} \qquad ( 14 )$$

In addition, the combined amplitude can be held constant at one using the relation in equation (15).

$${c}_{M} = \sqrt{{α}_{M}^{2} + {β}_{M}^{2}} \qquad ( 15 )$$

Therefore, any pattern of phase delays can be created across an aperture if, first, the apodization magnitude can be controlled for each element and, then, a π/2 group phase shift can be created between two transmissions. A transmission with the aperture function α_{M} and a cosine excitation followed by a transmission with the aperture function β_{M} and a sine wave excitation (π/2 phase shift from the first) creates the correct phasing for a one-way focus.

As explained above, a two-way focus can be created synthetically with four transmit/receive events to achieve each combination of sine and cosine apertures. To achieve the correct phase delays on receive, the procedure must be repeated as for transmit by applying the correct aperture apodization functions and shift the received signals by π/2 between two receive events.

However, as noted above, four transmit/receive events are needed because an equivalent wavefront in the elevation dimension must be transmitted for both receive apertures to create the correct phasing on receive. This notion is supported when considering how the field is modelled. Using an impulse response method, the receive voltage is modelled by convolving the excitation (vₑ(t)) with the scattering function of the field (fₛ(x₁)) and the spatial impulse response of both the transmit and receive aperture (hₜₓ(x₁, t, fₒₚ), hᵣₓ(x₁, t, fₒₚ)),

$${v}_{r} ( {x}_{1} , t ) = {v}_{e} ( t ) * {f}_{s} ( {x}_{1} ) * {h}_{t x} ( {x}_{1} , t , {f}_{op} ) * {h}_{r x} ( {x}_{1} , t , {f}_{op} ) \qquad ( 16 )$$

where x₁ represents a given field point and fₒₚ is the operating frequency. And the excitation signal is described by,

$${v}_{e} ( t ) = A * cos ( 2 π {f}_{o p} t + θ ) \qquad ( 17 )$$

Where A represents the excitation amplitude and θ is the phase of the excitation.

The spatial impulse responses of the transmit and receive aperture can be considered separately. By synthetically combining the apodized sine and cosine apertures, two spatial impulse responses are created, denoted with the subscript a and b, each with an associated excitation signal (18, 19).

$${v}_{e , a} ( t ) = A * cos ( 2 π {f}_{op} t + θ ) \qquad ( 18 )$$

$${v}_{e , b} ( t ) = A * cos ( 2 π {f}_{op} t + θ - \frac{π}{2} ) \qquad ( 19 )$$

In total, four combinations of excitation signal and spatial impulse response are required to synthetically create the quadrature apertures on both transmit and receive. Each of the four receive signals (v_{r,n}(x₁, t, fₒₚ) requires a unique combination of transmit excitation, transmit spatial impulse response, and receive spatial impulse response (which can be delayed by π/2 to emulate a sine aperture). The four receive signals are added to create the synthetic quadrature aperture with a two-way focus.

$${v}_{r , 1} ( {x}_{1} , t , {f}_{op} ) = {v}_{e , a} ( t ) * {h}_{a , tx} ( {x}_{1} , t , {f}_{op} ) * {h}_{a , r x} ( {x}_{1} , t , {f}_{op} ) * {f}_{s} ( {x}_{1} ) \qquad ( 20 a )$$

$${v}_{r , 2} ( {x}_{1} , t , {f}_{op} ) = {v}_{e , b} ( t ) * {h}_{b , tx} ( {x}_{1} , t , {f}_{op} ) * {h}_{a , r x} ( {x}_{1} , t , {f}_{op} ) * {f}_{s} ( {x}_{1} ) \qquad ( 20 b )$$

$${v}_{r , 3} ( {x}_{1} , t , {f}_{op} ) = {v}_{e , a} ( t ) * {h}_{a , t x} ( {x}_{1} , t , {f}_{op} ) * {h}_{b , rx} ( {x}_{1} , t - \frac{1}{4 \cdot {f}_{op}} , {f}_{op} ) * {f}_{s} ( {x}_{1} ) \qquad ( 20 c )$$

$${v}_{r , 4} ( {x}_{1} , t , {f}_{op} ) = {v}_{e , b} ( t ) * {h}_{b , tx} ( {x}_{1} , t , {f}_{op} ) * {h}_{b , rx} ( {x}_{1} , t - \frac{1}{4 \cdot {f}_{o p}} , {f}_{op} ) * {f}_{s} ( {x}_{1} ) \qquad ( 20 d )$$

$${v}_{r , total} ( {x}_{1} , t , {f}_{op} ) = {\sum }_{i = 1}^{4} {v}_{r , n} ( {x}_{1} , t , {f}_{op} ) \qquad ( 21 )$$

$${v}_{r , 2} ( {x}_{1} , t , {f}_{op} ) \neq {v}_{r , 3} ( {x}_{1} , t , {f}_{op} ) \qquad ( 22 )$$

### Example 8: Theory of Phase Delay Control in Synthetic Transmit/Receive Quadrature Excitation Fresnel Focusing for Tissue Harmonic Imaging

The aforementioned example embodiments are typically implemented using the same operational frequency on transmit and receive. It has been shown using conventional ultrasound diagnostic transducers that harmonic imaging improves contrast and resolution over standard imaging that transmits and receives at the same frequency.

Traditional tissue harmonic imaging (THI) may be accomplished either using a filtered technique where only one transmit is required or a pulse-inversion method where two transmits are required which are 180 degrees out of phase. Fresnel tissue harmonic imaging (FTHI) may also be accomplished with either a filtered technique or pulse inversion technique.

### Filtered Fresnel Tissue Harmonic Imaging

If using the filtered technique, four transmits are still required. However, unlike traditional THI where the filtering typically occurs on the received beamformed signal, FTHI filtering starts at the receive aperture where the Fresnel pattern is determined by the harmonic frequency in addition to filtering on the received beamformed signal.

$${v}_{r , 1} ( {x}_{1} , t , {f}_{op} ) = A \cdot cos ( 2 π {f}_{op} \cdot t + θ ) * {h}_{a , t x} ( {x}_{1} , t , {f}_{op} ) * {h}_{a , r x} ( {x}_{1} , t , 2 {f}_{op} ) * {f}_{s} ( {x}_{1} ) \qquad ( 23 a )$$

$${v}_{r , 2} ( {x}_{1} , t , {f}_{op} ) = A \cdot cos ( 2 π {f}_{op} \cdot t + θ - \frac{π}{2} ) * {h}_{b , tx} ( {x}_{1} , t , {f}_{op} ) * {h}_{a , r x} ( {x}_{1} , t , 2 {f}_{op} ) * {f}_{s} ( {x}_{1} ) \qquad ( 23 b )$$

$${v}_{r , 3} ( {x}_{1} , t , {f}_{op} ) = A \cdot cos ( 2 π {f}_{o p} \cdot t + θ ) * {h}_{a , t x} ( {x}_{1} , t , {f}_{o p} ) * {h}_{b , rx} ( {x}_{1} , t - \frac{1}{8 \cdot {f}_{o p}} , 2 {f}_{o p} ) * {f}_{s} ( {x}_{1} ) \qquad ( 23 c )$$

$${v}_{r , 4} ( {x}_{1} , t , {f}_{op} ) = A \cdot cos ( 2 π {f}_{op} \cdot t + θ - \frac{π}{2} ) * {h}_{b , tx} ( {x}_{1} , t , {f}_{op} ) * {h}_{b , rx} ( {x}_{1} , t - \frac{1}{8 \cdot {f}_{op}} , 2 {f}_{op} ) * {f}_{s} ( {x}_{1} ) \qquad ( 23 d )$$

$${v}_{r , total} ( {x}_{1} , t , {f}_{op} ) = {\sum }_{i = 1}^{4} {v}_{r , n} ( {x}_{1} , t , {f}_{op} ) \qquad ( 24 )$$

$${v}_{r , 2} ( {x}_{1} , t , {f}_{op} ) \neq {ν}_{r , 3} ( {x}_{1} , t , {f}_{o p} ) ) \qquad ( 25 )$$

$$A \cdot cos ( 2 π {f}_{op} \cdot t + θ - \frac{π}{2} ) * {h}_{b , tx} ( {x}_{1} , t , {f}_{op} ) * {h}_{a , r x} ( {x}_{1} , t , 2 {f}_{op} ) * {f}_{s} ( {x}_{1} ) \neq A \cdot cos ( 2 π {f}_{op} \cdot t + θ ) * {h}_{a , t x} ( {x}_{1} , t , {f}_{op} ) * {h}_{b , rx} ( {x}_{1} , t - \frac{1}{8 \cdot {f}_{op}} , 2 {f}_{op} ) * {f}_{s} ( {x}_{1} ) \qquad ( 26 )$$

The first four equations which represent the four transmits have an additional variable ‘fₒₚ’ added to show that the Fresnel apertures for both transmit and receive are functions of the operational frequency ‘fₒₚ’. The operational frequency on receive is twice the operational frequency on transmit. Of course, the receive frequency may be varied based on where the harmonics are generated and does not have to be twice the transmit frequency. In transmit, two orthogonal apertures are used to produce the ideal phasing for a transmit aperture at ‘fₒₚ’. In receive, two orthogonal apertures are used to produce the ideal phasing for the harmonic frequency which is ‘2fₒₚ’ in this representation. Since the filtered FTHI uses a special receive aperture to focus on the harmonic frequency, the fundamental frequency suppression is better than traditional THI if the same received beamforming filters are applied. It is important to note that the number of transmits for filtered Fresnel THI can be reduced to one if an aperture is used that has both sine and cosine excitations as well as odd and even biases available simultaneously. In this case, the receive aperture is approximately twice the transmit frequency.

### Pulse-Inversion Fresnel Tissue Harmonic Imaging

Eight transmits are required if using pulse-inversion FTHI. This is because the received responses are summed together such that any energy at the fundamental frequency is eliminated and only received signal at the harmonics remains. Additional filtering may be used on the received signal to further isolate the harmonic energy of interest. Pulse-inversion FTHI also has the advantage over standard techniques in that the receive apertures are designed to focus at one frequency. The inverted transmit aperture may be applied using the bias lines or the excitation on the signal line. The eight equations below show that two transmit apertures are required to generate the ideal phasing. Similarly, two receive apertures are required to generate the ideal phasing for each transmit aperture. Therefore, four transmit-receive events are required to generate ideal phasing on both transmit and receive. This doubles to eight transmit-receive events for pulse-inversion FTHI since the inverted transmit also requires four transmit-receive events to generate ideal phasing in both transmit and receive.

$${v}_{r , 1} ( {x}_{1} , t , {f}_{op} ) = A \cdot cos ( 2 π {f}_{op} \cdot t + θ ) * {h}_{a , t x} ( {x}_{1} , t , {f}_{op} ) * {h}_{a , r x} ( {x}_{1} , t , 2 {f}_{op} ) * {f}_{s} ( {x}_{1} ) \qquad ( 27 a )$$

$${v}_{r , 2} ( {x}_{1} , t , {f}_{op} ) = A \cdot cos ( 2 π {f}_{op} \cdot t + θ - \frac{π}{2} ) * {h}_{b , tx} ( {x}_{1} , t , {f}_{op} ) * {h}_{a , r x} ( {x}_{1} , t , 2 {f}_{op} ) * {f}_{s} ( {x}_{1} ) \qquad ( 27 b )$$

$${v}_{r , 3} ( {x}_{1} , t , {f}_{op} ) = A \cdot cos ( 2 π {f}_{op} \cdot t + θ ) * {h}_{a , t x} ( {x}_{1} , t , {f}_{op} ) * {h}_{b , rx} ( {x}_{1} , t - \frac{1}{8 \cdot {f}_{op}} , 2 {f}_{op} ) * {f}_{s} ( {x}_{1} ) \qquad ( 27 c )$$

$${v}_{r , 4} ( {x}_{1} , t , {f}_{op} ) = A \cdot cos ( 2 π {f}_{op} \cdot t + θ - \frac{π}{2} ) * {h}_{b , tx} ( {x}_{1} , t , {f}_{op} ) * {h}_{b , rx} ( {x}_{1} , t - \frac{1}{8 \cdot {f}_{op}} , 2 {f}_{op} ) * {f}_{s} ( {x}_{1} ) \qquad ( 27 d )$$

$${v}_{r , 5} ( {x}_{1} , t , {f}_{o p} ) = - A \cdot cos ( 2 π {f}_{op} \cdot t + θ ) * {h}_{a , t x} ( {x}_{1} , t , {f}_{op} ) * {h}_{a , r x} ( {x}_{1} , t , 2 {f}_{op} ) * {f}_{s} ( {x}_{1} ) \qquad ( 27 e )$$

$${v}_{r , 6} ( {x}_{1} , t , {f}_{o p} ) = - A \cdot cos ( 2 π {f}_{o p} \cdot t + θ - \frac{π}{2} ) * {h}_{b , tx} ( {x}_{1} , t , {f}_{o p} ) * {h}_{a , r x} ( {x}_{1} , t , 2 {f}_{o p} ) * {f}_{s} ( {x}_{1} ) \qquad ( 27 f )$$

$${v}_{r , 7} ( {x}_{1} , t , {f}_{o p} ) = - A \cdot cos ( 2 π {f}_{o p} \cdot t + θ ) * {h}_{a , t x} ( {x}_{1} , t , {f}_{o p} ) * {h}_{b , r x} ( {x}_{1} , t - \frac{1}{8 \cdot {f}_{op}} , 2 {f}_{op} ) * {f}_{s} ( {x}_{1} ) \qquad ( 27 g )$$

$${v}_{r , 8} ( {x}_{1} , t , {f}_{o p} ) = - A \cdot cos ( 2 π {f}_{o p} \cdot t + θ - \frac{π}{2} ) * {h}_{b , tx} ( {x}_{1} , t , {f}_{o p} ) * {h}_{b , r x} ( {x}_{1} , t - \frac{1}{8 \cdot {f}_{o p}} , 2 {f}_{o p} ) * {f}_{s} ( {x}_{1} ) \qquad ( 27 h )$$

$${v}_{r , total} ( {x}_{1} , t , {f}_{o p} ) = {\sum }_{i = 1}^{8} {v}_{r , n} ( {x}_{1} , t , {f}_{o p} ) \qquad ( 28 )$$

It is important to note that the number of transmits for pulse-inversion Fresnel THI can be reduced to two if an aperture is used that has both sine and cosine excitations as well as odd and even biases available simultaneously. In this case, the receive aperture is approximately twice the transmit frequency and the two transmits are opposites of each other (negative).

In some example implementations, the Fresnel aperture can be adjusted over time during the receive window to increase the focusing depth and account for frequency dependent attenuation. This compensation in the Fresnel aperture could be beneficial for applications requiring a high operating frequency (i.e. high resolution B-mode imaging or tissue harmonic imaging) because the attenuation is most detrimental at high frequencies and can shift the center frequency of a propagating pulse significantly. For example, in equations 27a-d, the constant fₒₚ in the receive aperture functions

$$( {h}_{a , r x} ( {x}_{1} , t , {f}_{o p} ) and {h}_{b , rx} ( {x}_{1} , t - \frac{1}{8 \cdot {f}_{op}} , 2 {f}_{op} ) )$$

could be replaced by f′ₒₚ(t) to capture the dynamically changing operating frequency. The function f′ₒₚ(t) could be determined with advanced knowledge of the frequency dependent attenuation in the tissue of interest. The attenuation could be measured across the bandwidth and depths of interest and stored, as a look up table or analytical formula, as part of a preset configuration file for the tissue.

In some example implementations, this concept may be applied to conventional B-mode imaging, instead of, or in addition to, tissue harmonic imaging. In some example implementations, the receive Fresnel aperture could be adjusted over time to only account for the increasing focusing depth while the waves are propagating back to the transducer. In these example implementations, the bias levels on receive could transition continuously rather than be held constant for the duration of the receive window.

Although, in some example implementations, the transition from a shallow receive focus to a deep receive focus could be continuous and based on attenuation, it will be understood that this is not a requirement. In some example implementations, a discrete pattern may be used where the transmit Fresnel pattern depends on fₒₚ and the receive Fresnel pattern depends on f′ₒₚ. In such an example implementation, the Fresnel patterns for the transmit and receive apertures are calculated based on different operating frequencies.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1. A system for performing ultrasound imaging, the system comprising:
a row-column ultrasound transducer comprising:
a two-dimensional array of ultrasound elements arranged along a plurality of rows and columns, each ultrasound element being capable of acoustic transduction when a bias is applied thereto, such that a phase of ultrasound waves emitted therefrom is dependent on a polarity of the bias;
a set of signal conductive paths, each signal conductive path being in electrical communication with signal electrodes of ultrasound elements residing along a respective column of the two-dimensional array; and
a set of bias conductive paths, each bias conductive path being in electrical communication with bias electrodes of ultrasound elements residing along a respective row of the two-dimensional array; and
control and processing circuitry operatively coupled to said signal conductive paths and said bias conductive paths, said control and processing circuitry comprising at least one processor and associated memory, said memory comprising instructions executable by said processor to perform, in a selected temporal order, a set of synthetic transmit and receive operations comprising:
a first transmit operation and first receive operation performed by:
while applying a first transmit bias aperture to said bias conductive paths, delivering a first set of transmit signals to said signal conductive paths; and
while applying a first receive bias aperture to said bias conductive paths, receiving a first set of receive signals from said signal conductive paths;
a second transmit operation and second receive operation performed by:
while applying the first transmit bias aperture to said bias conductive paths, delivering a second set of transmit signals to said signal conductive paths, the second set of transmit signals being generated according to a driving waveform that is in phase with a corresponding driving waveform employed to generate the first set of transmit signals;
while applying a second receive bias aperture to said bias conductive paths, receiving a second set of receive signals from said signal conductive paths; and
applying a time delay to the second set of received signals, the time delay corresponding to a phase delay of pi/2
a third transmit operation and third receive operation performed by:
while applying a second transmit bias aperture to said bias conductive paths, delivering a third set of transmit signals to said signal conductive paths, the third set of transmit signals being generated according to a driving waveform that is phase shifted, relative to a corresponding driving waveform employed to generate the first set of transmit signals, by pi/2; and
while applying the first receive bias aperture to said bias conductive paths, receiving a third set of receive signals from said signal conductive paths; and
a fourth transmit operation and fourth receive operation performed by:
while applying the second transmit bias aperture to said bias conductive paths, delivering a fourth set of transmit signals to said signal conductive paths, the fourth set of transmit signals being generated according to a driving waveform that is phase shifted, relative to a corresponding driving waveform employed to generate the first set of transmit signals, by pi/2;
while applying the second receive bias aperture to said bias conductive paths, receiving a fourth set of receive signals from said signal conductive paths; and
applying a time delay to the fourth set of received signals, the time delay corresponding to a phase delay of pi/2;
summing the first, second, third and fourth sets of receive signals to obtain a summed set of receive signals; and
beamforming the summed set of receive signals in the azimuth dimension to generate an image;
wherein the first set of transmit signals, the second set of transmit signals, the third set of transmit signals and the fourth set of transmit signals are selected such that the resulting plurality of ultrasound pulses generated therefrom spatially overlap with each other in a far field region;
wherein the first transmit bias aperture and the second transmit bias aperture are configured to generate a synthetic transmit Fresnel aperture when the set of synthetic transmit and receive operations are compounded; and
wherein the first receive bias aperture and the second receive bias aperture are configured to generate a synthetic receive Fresnel aperture when the set of synthetic transmit and receive operations are compounded.

2. The system according to claim 1 wherein said control and processing circuitry is configured such that the synthetic transmit Fresnel aperture and the synthetic receive Fresnel aperture are configured to generate different elevation foci.

3. The system according to claim 1 wherein said control and processing circuitry is configured such that the synthetic transmit Fresnel aperture and the synthetic receive Fresnel aperture are configured to generate common elevation foci.

4. The system according to claim 1 wherein said control and processing circuitry is configured such an amplitude associated with the synthetic transmit Fresnel aperture is equal for at least two rows of the synthetic transmit Fresnel aperture.

5. The system according to claim 1 wherein said control and processing circuitry is configured such an amplitude associated with the synthetic receive Fresnel aperture is equal for at least two rows of the synthetic receive Fresnel aperture.

6. The system according to claim 1 wherein said control and processing circuitry is configured such that the bias levels of the transmit bias apertures and the receive bias apertures are generated according to a discrete set of bias levels, the discrete set of bias levels comprising at least three distinct bias levels.

7. The system according to claim 6 wherein said control and processing circuitry is configured such that the bias levels of the first transmit bias aperture and the bias levels of the second transmit bias aperture are obtained from a lookup table, the lookup table associating, for each phase range of a plurality of phase ranges, a suitable first transmit aperture bias value selected from the discrete set of bias levels and a suitable second transmit aperture bias level selected from the discrete set of bias levels, such that the synthetic transmit Fresnel aperture generated by the compounding of the transmit and receive operations approximates a desired transmit Fresnel aperture.

8. The system according to claim 6 wherein said control and processing circuitry is configured such that the bias levels of the first receive bias aperture and the bias levels of the second receive bias aperture are obtained from a lookup table, the lookup table associating, for each phase range of a plurality of phase ranges, a suitable first receive aperture bias value selected from the discrete set of bias levels and a suitable second receive aperture bias level selected from the discrete set of bias levels, such that the synthetic receive Fresnel aperture generated by the compounding of the transmit and receive operations approximates a desired ideal receive transmit Fresnel aperture.

9. The system according to claim 1 wherein said control and processing circuitry is configured such that the set of synthetic transmit and receive operations are performed in a sequence that minimizes switching between the bias apertures.

10. The system according to claim 1 wherein said control and processing circuitry is configured such that the first set of transmit signals, the second set of transmit signals, the third set of transmit signals and the fourth set of transmit signals are generated according to a time-delay aperture for focusing the resulting plurality of ultrasound pulses generated therefrom along a selected azimuth image line.

11. The system according to claim 1 wherein said control and processing circuitry is configured such that the first set of transmit signals, the second set of transmit signals, the third set of transmit signals and the fourth set of transmit signals are selected such that the resulting plurality of ultrasound pulses generated therefrom have respective wavefronts suitable for performing coherent compound imaging in the azimuth direction.

12. The system according to claim 11 wherein the set of synthetic transmit and receive operations is a first set of synthetic transmit and receive operations, and wherein said control and processing circuitry is configured such that:
the following additional steps are performed one or more times:
performing an additional set of synthetic transmit and receive operations employing an additional first set of transmit signals, an additional second set of transmit signals, an additional third set of transmit signals and an additional fourth set of transmit signals selected such that the resulting plurality of additional ultrasound pulses generated therefrom have respective wavefronts suitable for performing coherent compound imaging in the azimuth direction;
wherein the resulting additional first, additional second, additional third and additional fourth sets of receive signals are summed with the first, second, third and fourth sets of receive signals to obtain the summed set of receive signals.

13. The system according to claim 12 wherein a focal location corresponding to at least one of the synthetic transmit Fresnel aperture and the synthetic receive Fresnel aperture is different for at least two sets of synthetic transmit and receive operations.

14. The system according to claim 12 wherein the synthetic transmit Fresnel aperture and the synthetic receive Fresnel aperture corresponding to each set of synthetic transmit and receive operations are Fresnel sub-apertures.

15. The system according to claim 1 wherein the row-column ultrasound transducer comprises an electrostrictive material.

16. The system according to claim 1 wherein the row-column ultrasound transducer is formed from an array of capacitive micromachined ultrasound transducer elements.

17. The system according to claim 1 wherein each receive bias aperture is calculated for a harmonic of a transmit frequency to perform Fresnel tissue harmonic imaging.

18. The system according to claim 1 wherein the set of transmit operations are a first set of transmit operations, and wherein an additional set of transmit operations are employed with an inverted phase, corresponding to a phase delay of pi, relative to the first set of transmit operations, to perform pulse inversion Fresnel tissue harmonic imaging.

19. The system according to claim 1 wherein at least one receive bias aperture is time dependent and calculated based on a dynamic focusing depth.

20. The system according to claim 1 wherein at least one receive bias aperture is time dependent and is calculated based on a known dependence of frequency on depth.

21. The system according to any claim 1 wherein at least one receive bias aperture is calculated based on a receive focusing depth.

22. The system according to claim 1 wherein at least one receive bias aperture is calculated based on a known dependence of frequency on depth.

23. The system according to claim 1 wherein at least one receive bias aperture is calculated based on a frequency of interest.

24. A system for generating focused ultrasound, the system comprising:
a row-column ultrasound transducer comprising:
a two-dimensional array of ultrasound elements arranged in a diamond pattern defining a set of rows and columns, such that for a given transducer element, a row associated with the given transducer element extends along a first axis connecting a first pair of opposing vertices of the given transducer element, and a column associated with the given transducer element extends along a second axis connecting a second pair of opposing vertices of the given transducer element, each ultrasound element being capable of acoustic transduction when a bias is applied thereto, such that a phase of ultrasound waves emitted therefrom is dependent on a polarity of the bias;
a set of signal conductive paths, each signal conductive path extending along a respective column of the two-dimensional array and being in electrical communication with signal electrodes of ultrasound elements of the respective column, said set of signal conductive paths comprising a set of odd signal conductive paths and a set of even signal conductive paths, wherein each even signal conductive path extends along a respective even column of the diamond pattern and each odd signal conductive path extends along a respective odd column of the diamond pattern; and
a set of bias conductive paths, each bias conductive path extending along a respective row of the two-dimensional array and being in electrical communication with bias electrodes of ultrasound elements of the respective row, said set of bias conductive paths comprising a set of odd bias conductive paths and a set of even bias conductive paths, wherein each even bias conductive path extends along a respective even row of the diamond pattern and each odd bias conductive path extends along a respective odd row of the diamond pattern; and
control and processing circuitry operatively coupled to said set of signal conductive paths and said set of bias conductive paths, said control and processing circuitry comprising at least one processor and associated memory, said memory comprising instructions executable by said processor to perform a transmit operation comprising:
applying a first bias aperture to said set of odd bias conductive paths and a second bias aperture to said set of even bias conductive paths, the first bias aperture comprising a set of first bias values and the second bias aperture comprising a corresponding set of second bias values; and
while applying the first bias aperture and the second bias aperture, delivering, to said set of odd signal conductive paths and said set of even signal conductive paths, a set of transmit signals defined according to a time-delay aperture, wherein set of transmit signals are provided to said set of even signal conductive paths in quadrature;
the set of first bias values and the corresponding set of second bias values being configured such that the transmit operation results in the generation of a synthetic Fresnel aperture.

25. A system for generating focused ultrasound, the system comprising:
a row-column ultrasound transducer comprising:
a two-dimensional array of ultrasound elements arranged along a plurality of rows and columns;
within each row, each ultrasound element comprising a first sub-element and a second sub-element residing laterally adjacent to one another, each sub-element being capable of acoustic transduction when a bias is applied thereto, such that a phase of ultrasound waves emitted therefrom is dependent on a polarity of the bias, such that each column of the two-dimensional array comprises a first sub-column of first sub-elements and a second sub-column column of second sub-elements;
a set of signal conductive paths, each signal conductive path being in electrical communication with signal electrodes of ultrasound elements residing along a respective sub-column of the two-dimensional array, such that the first sub-column and second sub-column within a given column are separately addressable by respective first and second signal conductive paths; and
a set of bias conductive path pairs, each bias conductive path pair comprising a first bias conductive path and a second bias conductive path that both extend along or adjacent to a given row of the two-dimensional array such that said first bias conductive path is configured to apply a respective bias to the first sub-elements with the given row and said second bias conductive path is configured to apply a respective bias to the second sub-elements within the given row; and
control and processing circuitry operatively coupled to said signal conductive paths and said set of bias conductive path pairs, said control and processing circuitry comprising at least one processor and associated memory, said memory comprising instructions executable by said processor to perform a transmit operation comprising:
applying a first bias aperture to said first bias conductive paths and a second bias aperture to said second bias conductive paths, the first bias aperture comprising a set of first bias values and the second bias aperture comprising a corresponding set of second bias values; and
while applying the first bias aperture and the second bias aperture, delivering a set of transmit signals to said signal conductive paths according to a time-delay aperture, such that the time-delay aperture defines transmit beamforming time delays associated with each column of the two-dimensional array, and such that for a given column having a respective beamforming time delay associated therewith, the transmit signal delivered to the first sub-column and the second sub-column have a common temporal envelope temporally delayed according to the beamforming delay, and wherein a waveform phase of the second sub-column is shifted, relative to that of the first sub-column, by pi/2, the first sub-column and the second sub-column of a given column thereby being delivered beamformed transmit signals in quadrature;
the set of first bias values and the corresponding set of second bias values being configured such that the transmit operation results in the generation of a Fresnel aperture.

26. The system according to claim 25 wherein said control and processing circuitry is configured such an amplitude associated with the Fresnel aperture is equal for at least two rows of the Fresnel aperture.

27. The system according to claim 25 wherein said control and processing circuitry is configured such that the set of first bias values and the set of second bias values are generated according to a discrete set of bias levels, the discrete set of at bias levels comprising at least three bias levels.

28. The system according to claim 27 wherein said control and processing circuitry is configured such that each first bias value of the first bias aperture and each corresponding second bias value of the second bias aperture are obtained by:
generating a lookup table associating, for each phase range of a plurality of phase ranges, a suitable first bias value selected from the discrete set of bias levels and a suitable second bias level selected from the discrete set of bias levels, such that the transmit operation applying the suitable first bias value and the suitable second bias level to ultrasound elements within a given row of the two-dimensional array results in a phase that lies within the phase range.

29. The system according to claim 25 wherein the row-column ultrasound transducer comprises an electrostrictive material.

30. The system according to claim 25 wherein the row-column ultrasound transducer is formed from an array of capacitive micromachined ultrasound transducer elements.

31. A method of performing ultrasound imaging, the method comprising:
providing a row-column ultrasound transducer comprising:
a two-dimensional array of ultrasound elements arranged along a plurality of rows and columns, each ultrasound element being capable of acoustic transduction when a bias is applied thereto, such that a phase of ultrasound waves emitted therefrom is dependent on a polarity of the bias;
a set of signal conductive paths, each signal conductive path being in electrical communication with signal electrodes of ultrasound elements residing along a respective column of the two-dimensional array; and
a set of bias conductive paths, each bias conductive path being in electrical communication with bias electrodes of ultrasound elements residing along a respective row of the two-dimensional array; and
performing, in a selected temporal order, a set of synthetic transmit and receive operations comprising:
a first transmit operation and first receive operation performed by:
while applying a first transmit bias aperture to said bias conductive paths, delivering a first set of transmit signals to said signal conductive paths; and
while applying a first receive bias aperture to said bias conductive paths, receiving a first set of receive signals from said signal conductive paths;
a second transmit operation and second receive operation performed by:
while applying the first transmit bias aperture to said bias conductive paths, delivering a second set of transmit signals to said signal conductive paths, the second set of transmit signals being generated according to a driving waveform that is in phase with a corresponding driving waveform employed to generate the first set of transmit signals;
while applying a second receive bias aperture to said bias conductive paths, receiving a second set of receive signals from said signal conductive paths; and
applying a time delay to the second set of received signals, the time delay corresponding to a phase delay of pi/2
a third transmit operation and third receive operation performed by:
while applying a second transmit bias aperture to said bias conductive paths, delivering a third set of transmit signals to said signal conductive paths, the third set of transmit signals being generated according to a driving waveform that is phase shifted, relative to a corresponding driving waveform employed to generate the first set of transmit signals, by pi/2; and
while applying the first receive bias aperture to said bias conductive paths, receiving a third set of receive signals from said signal conductive paths; and
a fourth transmit operation and fourth receive operation performed by:
while applying the second transmit bias aperture to said bias conductive paths, delivering a fourth set of transmit signals to said signal conductive paths, the fourth set of transmit signals being generated according to a driving waveform that is phase shifted, relative to a corresponding driving waveform employed to generate the first set of transmit signals, by pi/2;
while applying the second receive bias aperture to said bias conductive paths, receiving a fourth set of receive signals from said signal conductive paths; and
applying a time delay to the fourth set of received signals, the time delay corresponding to a phase delay of pi/2;
summing the first, second, third and fourth sets of receive signals to obtain a summed set of receive signals; and
beamforming the summed set of receive signals in the azimuth dimension to generate an image;
wherein the first set of transmit signals, the second set of transmit signals, the third set of transmit signals and the fourth set of transmit signals are selected such that the resulting plurality of ultrasound pulses generated therefrom spatially overlap with each other in a far field region;
wherein the first transmit bias aperture and the second transmit bias aperture are configured to generate a synthetic transmit Fresnel aperture when the set of synthetic transmit and receive operations are compounded; and
wherein the first receive bias aperture and the second receive bias aperture are configured to generate a synthetic receive Fresnel aperture when the set of synthetic transmit and receive operations are compounded.

32-46. (canceled)

47. A method of generating focused ultrasound, the method comprising:
providing a row-column ultrasound transducer comprising:
a two-dimensional array of ultrasound elements arranged in a diamond pattern defining a set of rows and columns, such that for a given transducer element, a row associated with the given transducer element extends along a first axis connecting a first pair of opposing vertices of the given transducer element, and a column associated with the given transducer element extends along a second axis connecting a second pair of opposing vertices of the given transducer element, each ultrasound element being capable of acoustic transduction when a bias is applied thereto, such that a phase of ultrasound waves emitted therefrom is dependent on a polarity of the bias;
a set of signal conductive paths, each signal conductive path extending along a respective column of the two-dimensional array and being in electrical communication with signal electrodes of ultrasound elements of the respective column, said set of signal conductive paths comprising a set of odd signal conductive paths and a set of even signal conductive paths, wherein each even signal conductive path extends along a respective even column of the diamond pattern and each odd signal conductive path extends along a respective odd column of the diamond pattern; and
a set of bias conductive paths, each bias conductive path extending along a respective row of the two-dimensional array and being in electrical communication with bias electrodes of ultrasound elements of the respective row, said set of bias conductive paths comprising a set of odd bias conductive paths and a set of even bias conductive paths, wherein each even bias conductive path extends along a respective even row of the diamond pattern and each odd bias conductive path extends along a respective odd row of the diamond pattern; and
applying a first bias aperture to said set of odd bias conductive paths and a second bias aperture to said set of even bias conductive paths, the first bias aperture comprising a set of first bias values and the second bias aperture comprising a corresponding set of second bias values; and
while applying the first bias aperture and the second bias aperture, delivering, to said set of odd signal conductive paths and said set of even signal conductive paths, a set of transmit signals defined according to a time-delay aperture, wherein set of transmit signals are provided to said set of even signal conductive paths in quadrature;
wherein the set of first bias values and the corresponding set of second bias values are configured such that the transmit operation results in the generation of a synthetic Fresnel aperture.

48. A method of generating focused ultrasound, the method comprising:
providing a row-column ultrasound transducer comprising:
a two-dimensional array of ultrasound elements arranged along a plurality of rows and columns;
within each row, each ultrasound element comprising a first sub-element and a second sub-element residing laterally adjacent to one another, each sub-element being capable of acoustic transduction when a bias is applied thereto, such that a phase of ultrasound waves emitted therefrom is dependent on a polarity of the bias, such that each column of the two-dimensional array comprises a first sub-column of first sub-elements and a second sub-column column of second sub-elements;
a set of signal conductive paths, each signal conductive path being in electrical communication with signal electrodes of ultrasound elements residing along a respective sub-column of the two-dimensional array, such that the first sub-column and second sub-column within a given column are separately addressable by respective first and second signal conductive paths; and
a set of bias conductive path pairs, each bias conductive path pair comprising a first bias conductive path and a second bias conductive path that both extend along or adjacent to a given row of the two-dimensional array such that said first bias conductive path is configured to apply a respective bias to the first sub-elements with the given row and said second bias conductive path is configured to apply a respective bias to the second sub-elements within the given row; and
applying a first bias aperture to said first bias conductive paths and a second bias aperture to said second bias conductive paths, the first bias aperture comprising a set of first bias values and the second bias aperture comprising a corresponding set of second bias values; and
while applying the first bias aperture and the second bias aperture, delivering a set of transmit signals to said signal conductive paths according to a time-delay aperture, such that the time-delay aperture defines transmit beamforming time delays associated with each column of the two-dimensional array, and such that for a given column having a respective beamforming time delay associated therewith, the transmit signal delivered to the first sub-column and the second sub-column have a common temporal envelope temporally delayed according to the beamforming delay, and wherein a waveform phase of the second sub-column is shifted, relative to that of the first sub-column, by pi/2, the first sub-column and the second sub-column of a given column thereby being delivered beamformed transmit signals in quadrature;
wherein the set of first bias values and the corresponding set of second bias values are configured such that the transmit operation results in the generation of a Fresnel aperture.

49-53. (canceled)
